(19) United States
(12) Patent Application Publication
(10) Pub. No.: US 2024/0335402 A1
(43) Pub. Date: Oct. 10, 2024

(54) BIODEGRADABLE ANTI-INFECTIVE FORMULATIONS

(71) Applicant: New Mexico Tech University Research Park Corporation, Socorro, NM (US)

(72) Inventors: Snezna ROGELJ, Socorro, NM (US); Danielle Nicole TURNER, Socorro, NM (US)

(21) Appl. No.: 18/625,785

(22) Filed: Apr. 3, 2024

Related U.S. Application Data
(60) Provisional application No. 63/494,039, filed on Apr. 4, 2023.

(51) Int. Cl. A61K 31/14; A61K 31/131; A61K 47/12

(52) U.S. Cl. CPC A61K 31/14; A61K 31/131; A61K 47/12

(57) ABSTRACT

The present disclosure provides pharmaceutical compositions for treating fungal, viral, and/or bacterial infections. The pharmaceutical compositions of the disclosure can comprise a cationic surfactant, a chelating agent, a pH modifier, and a solvent. The pharmaceutical compositions of the disclosure can be used to treat drug-sensitive or multidrug-resistant bacterial, viral, and/or fungal infections. In some embodiments, the chelating agent can be biodegradable.

### CROSS-REFERENCE

This application claims the benefit of U.S. Provisional Application No. 63/494,039, filed Apr. 4, 2023, which is incorporated herein by reference in its entirety.

### BACKGROUND

Microorganisms can cause substantial hygiene and health problems. Disinfection to kill the microorganisms and/or inhibit the growth or regrowth of the microorganisms is required in industries (e.g., food industry), health services, household, and also in clinical settings. Most disinfectants contain alcohol, strong oxidizing reagents, and/or toxic compounds. They may also be non-biodegradable, have limited in vivo applications, and have a narrow spectrum of bioactivity for potential clinical applications.

### INCORPORATION BY REFERENCE

Each patent, publication, and non-patent literature cited in the application is hereby incorporated by reference in its entirety as if each was incorporated by reference individually.

### SUMMARY OF THE DISCLOSURE

Provided herein are antibacterial, antifungal, and antiviral formulations that are non-toxic, nonflammable, and biodegradable. The formulations provided herein can work against viruses and drug sensitive and multiple drug-resistant (or multidrug-resistant) pathogens.

In some embodiments, the present disclosure provides a composition comprising, in a mixture: a) a cationic surfactant, wherein the cationic surfactant is a cetyltrimethylammonium halide; b) a chelating agent, wherein the chelating agent is N,N-Dicarboxymethyl glutamic acid (GLDA) or a salt thereof; c) a pH modifier; and d) a solvent.

In some embodiments, the present disclosure provides a method of killing a microorganism comprising contacting the microorganism with an effective amount of a composition, wherein the composition comprises, in a mixture: a) a cationic surfactant, wherein the cationic surfactant is a cetyltrimethylammonium halide; b) a chelating agent, wherein the chelating agent is N,N-Dicarboxymethyl glutamic acid (GLDA) or a salt thereof; c) a pH modifier; and d) a solvent. In some embodiments, the microorganism is a bacterium. In some embodiments, the microorganism is a fungus. In some embodiments, the fungus is a mold. In some embodiments, the microorganism is a yeast. In some embodiments, the microorganism is a spore. In some embodiments, the microorganism is a virus.

In some embodiments, the present disclosure provides a method of treating an infection comprising administering to a subject in need thereof a therapeutically-effective amount of a pharmaceutical composition, wherein the pharmaceutical composition comprises, in unit dosage form: a) a cationic surfactant, wherein the cationic surfactant is a cetyltrimethylammonium halide; b) a chelating agent, wherein the chelating agent is N,N-Dicarboxymethyl glutamic acid (GLDA) or a pharmaceutically-acceptable salt thereof; c) a pH modifier; and d) a solvent.

In some embodiments, the infection is caused by a bacterium. In some embodiments, the infection is caused by a fungus. In some embodiments, the fungus is a mold. In some embodiments, the infection is caused by a yeast. In some embodiments, the infection is caused by a spore. In some embodiments, the infection is caused by a virus.

In some embodiments, the present disclosure provides a method comprising contacting a surface of an object with a formulation, wherein the formulation comprises, in a mixture: a) a cationic surfactant, wherein the cationic surfactant is a cetyltrimethylammonium halide; b) a chelating agent, wherein the chelating agent is N,N-Dicarboxymethyl glutamic acid (GLDA) or a salt thereof; c) a pH modifier; and d) a solvent. In some embodiments, the contacting disinfects the surface. In some embodiments, contacting protect a surface from infection. In some embodiments, an infectious agent is present on the surface. In some embodiments, an infectious agent is present on the surface and the contacting substantially eliminates the infectious agent. In some embodiments, a fungus is present on the surface. In some embodiments, the fungus is a mold. In some embodiments, a yeast is present on the surface. In some embodiments, a virus is present on the surface. In some embodiments, a spore is present on the surface.

In some embodiments, the cetyltrimethylammonium halide is cetyltrimethylammonium chloride. In some embodiments, the cetyltrimethylammonium halide is cetyltrimethylammonium bromide. In some embodiments, the cetyltrimethylammonium halide is cetyltrimethylammonium iodide. In some embodiments, the chelating agent is N,N-Dicarboxymethyl glutamic acid tetrasodium salt. In some embodiments, the cationic surfactant is present in the mixture at a concentration of about 0.2 mM to about 50 mM. In some embodiments, the cationic surfactant is present in the mixture at a concentration of about 1-5 mM. In some embodiments, the cationic surfactant is present in the mixture at a concentration of about 2 mM. In some embodiments, the cationic surfactant is present in the mixture at a concentration of about 3 mM. In some embodiments, the chelating agent is present in the mixture at a concentration of about 1 mM to about 200 mM. In some embodiments, the chelating agent is present in the mixture at a concentration of about 20 mM. In some embodiments, the chelating agent is present in the mixture at a concentration of about 26 mM. In some embodiments, the chelating agent is present in the mixture at a concentration of about 50 mM. In some embodiments, the pH modifier comprises acetic acid. In some embodiments, the pH modifier is present in the mixture at a concentration of about 1 mM to about 150 mM. In some embodiments, the pH modifier is present in the mixture at a concentration of about 10 mM. In some embodiments, the pH modifier is present in the mixture at a concentration of about 15.6 mM. In some embodiments, the solvent is present in the mixture in an amount of about 96 wt % to 98.9 wt %. In some embodiments, the composition is substantially free of enzymes. In some embodiments, the composition is substantially free of oxidant. In some embodiments, the solvent is water.

In some embodiments, the present disclosure provides a method of treating a wound in a subject, comprising administering to a subject in need thereof a therapeutically-effective amount of a pharmaceutical composition, wherein the pharmaceutical composition comprises, in unit dosage form: a) a cationic surfactant, wherein the cationic surfactant is a cetyltrimethylammonium halide; b) a chelating agent, wherein the chelating agent is N,N-Dicarboxymethyl glutamic acid (GLDA) or a pharmaceutically-acceptable salt thereof; c) a pH modifier; and d) a solvent.

### BRIEF DESCRIPTION OF THE FIGURES

FIG. 1A shows the P. aeruginosa ATCC 2114-infected MHA plate treated with MD (F1), CTAC (F2), MG (F3) and MGD (F4) after an incubation time of 24 hours.

FIG. 1B shows the Listeria meningitides-infected MHA plate treated with MD (F1), CTAC (F2), MG (F3) and MGD (F4) after an incubation time of 24 hours.

FIG. 1C shows the Burkholderia cepacia ATCC 10856-infected MHA plate treated with MD (F1), CTAC (F2), MG (F3) and MGD (F4) after an incubation time of 24 hours.

FIG. 1D shows the Burkholderia thailandesis E426-infected MHA plate treated with MD (F1), CTAC (F2), MG (F3) and MGD (F4) after an incubation time of 24 hours.

FIG. 1E shows the S. aureus 29568-infected MHA plate treated with MD (F1), CTAC (F2), MG (F3) and MGD (F4) after an incubation time of 24 hours.

FIG. 1F shows the Bacillus cereus ATCC 14579-infected MHA plate treated with MD (F1), CTAC (F2), MG (F3) and MGD (F4) after an incubation time of 24 hours.

FIG. 1G shows the Staph aureus 29568-infected MHA plate treated with MD (F1), CTAC (F2), MG (F3) and MGD (F4) after an incubation time of 24 hours.

FIG. 1H shows the Bacillus cereus ATCC 14579-infected MHA plate treated with MD (F1), CTAC (F2), MG (F3) and MGD (F4) after an incubation time of 24 hours.

FIG. 1I shows the Burkholderia cepacia ATCC 10856-infected MHA plate treated with MD (F1), CTAC (F2), MG (F3) and MGD (F4) after an incubation time of 24 hours.

FIG. 1J shows the Burkholderia cepacia ATCC 10856-infected MHA plate treated with MD (F1), CTAC (F2), MG (F3) and MGD (F4) after an incubation time of 48 hours.

FIG. 1K shows that MG formulation and MGD formulation were equally effective against multidrug-resistant P. aeruginosa ATCC 2114 and MRSA BAA 44.

FIG. 1L shows the photograph of the plate after 3 days and the MG formulation was equally effective against Clostridium sporogenes as the MD formulation.

FIGS. 2A and 2B show both MG formulation and MGD formulation were effective against multidrug-resistant yeast C. neoformans and C. auris.

FIGS. 3A, 3B, and 3C show comparisons of MG treated wells and control wells (no MG treatment) after 24 hours, 8 days, and 2 months.

FIG. 4A shows the toxicity of chlorohexidine gluconate 0.12% oral rinse against HeLa cells. Chlorohexidine gluconate 0.12% oral rinse had an IC₅₀ (GI₅₀) of 0.402% v/v of the compound.

FIG. 4B shows the toxicity of MG against HeLa cells. MG had an IC₅₀ (GI₅₀) of 0.039% v/v of the compound.

FIG. 5A shows the toxicity of chlorohexidine gluconate 0.12% oral rinse against ARPE-19 cells. Chlorohexidine gluconate 0.12% oral rinse had an IC₅₀ (GI₅₀) of 0.658% v/v of the compound.

FIG. 5B shows the toxicity of MG against ARPE-19 cells.

FIG. 6A shows the killing activity of against Botrytis cinera ATCC 20599. FIG. 6B shows the killing activity of MG1 and MGI1 against Aspergillus niger. FIG. 6C shows the killing activity of MG1 and MGI1 against Cryptococcus neoformans.

FIG. 7A shows MRSA-infected MHA plate at 24 hours after treatment with MG or Mupirocin formulations. FIG. 7B shows MRSA-infected MHA plate at 2 days after treatment with MG or Mupirocin formulations. FIG. 7C shows MRSA-infected MHA plate at 8 days after treatment with MG or Mupirocin formulations.

FIGS. 8A-8C show killing activity to PME resistant pathogen S. maltophilia.

FIG. 9 shows a comparison of E. coli on MHA plates with MicroorGONE MG1 (MG1) treatment or no MG1 treatment.

FIG. 10 shows a comparison of E. coli on MHA plates with HCG treatment, no treatment, or HIMM treatment.

FIG. 11A shows Bacillus subtilis-infected plate with MG or MGT treatment. FIG. 11B shows S. maltophilia-infected plate with MG or MGT treatment. FIG. 11C shows C. albicans and MRSA-infected plate with MG1 or MG1T treatment. FIG. 11D shows P. aeruginosa and A. baumannii-infected plate with MG1 or MG1T treatment. FIG. 11E shows C. albicans-infected plate treated with MG, MG1T, MG0.5T, MG0.25T, MG0.12T, MG0.06T, MG0.03T, or CIT. FIG. 11F shows P. aeruginosa infected plate treated with MG, MGAA, MGT, and MGC. FIG. 11G shows P. aeruginosa infected plate treated with MG, MGT, MGAA, MGCu, and MGCuAA.

FIGS. 12A-12F show Candida albicans, Candida auris, Aspergillus niger, MRSA, A baumannii 15151, and MDR A. baumannii 1797 infected plate treated with HIMM, HC, MG1, and HCG, respectively. FIGS. 12G-12L show P. aeruginosa ATCC 27601, MDR P. aeruginosa ATCC BAA-2114, P. aeruginosa ATCC 15692, Mycobacterium smegmatis ATCC 607, E. coli 0157, and S. pyogenes Strep A infected plate treated with HIMM, HC, MG1, and HCG, respectively.

FIGS. 13A-13F show Burkholderia cepacia ATCC 10856, Proteus mirabilis, Vancomycin-resistant enterococcus VRE ATCC 51299, MDR Salmonella infantis AR0920, Yesenia pestis Kim 6, and Klebsiella pneumoniae CRE ATCC 1705 infected plate treated with HIMM, HC, and HCG, respectively. FIGS. 13G-13I show P. aeruginosa ATCC 15692, P. aeruginosa ATCC 2114, and P. aeruginosa 27853 infected plate treated with MG1, HIMM, HC, C, G, and HCG, respectively. FIG. 13J show Candida albicans ATCC 2655, MDR MRSA BAA 44, MDR A. baumannii ATCC 1797, and MDR P. aeruginosa ATCC 2114 growth pattern in presence of HIMM, HC, and HCG from left to right. FIG. 13K shows E. coli XL1 growth. FIG. 13L shows activity of HC formulation with reduced concentration of HIMM. FIG. 13M shows anti-bacterial activity of MG1, C, G, HC, HCG, and HIMM formulation.

FIG. 14 shows activity of MG formulation with ascorbic acid against C. albicans, MRSA and P. aeruginosa.

FIG. 15A shows the plate spotted with spores treated with MicroorGONE (“MG1”) and water (“H₂O”).

FIG. 15B shows an image of the plate treated with MG1 at different pH value.

FIGS. 16A-16D show images of the MHA plates inoculated with P. aeruginosa (FIG. 16A), S. aureus (FIG. 16B), B. cepacia (FIG. 16C), and C. albicans (FIG. 16D), that were spotted with MG1 solutions at different pH values.

FIG. 17A shows the quantification of different inoculums (PANEL A: pooled, PANEL B: B. cepacian, PANEL C: E. coli, PANEL D: P. aeruginosa, PANEL E: K. pneumonia, PANEL F: S. aureus, and PANEL G: C. albicans) using MHB before the treatments.

FIG. 17B shows the quantification of S. aureus stock inoculum (PANEL A) and after dilutions (PANELS B-D).

FIG. 17C shows the plates with S. aureus treated with MHB, PG, and PG+MG1 at different dilutions.

FIG. 18 shows the quantification of the pooled inoculums treated with different compositions for 2 days (PANEL A: Control, PANEL B: PG, PANEL C: PG+MG1, PANEL D: PG+1:10 MG1, PANEL E: PG+1:100 MG1).

FIG. 19 shows the quantification of the pooled inoculums treated with different compositions for 8 days (PANEL A: Control, PANEL B: PG, PANEL C: PG+MG1, PANEL D: PG+1:10 MG1, PANEL E: PG+1:100 MG1).

FIG. 20 PANEL A shows the quantification of S. aureus stock inoculum. FIG. 20 PANELS B-F show the quantification of S. aureus treated with different compositions (PANEL B: Control, PANEL C: PG, PANEL D: PG+MG1, PANEL E: PG+1:10 MG1, PANEL F: PG+1:100 MG1).

FIG. 21 PANEL A shows the quantification of C. albicans 1 stock inoculum. FIG. 21 PANELS B-D show the quantification of C. albicans 1 treated with different compositions (PANEL B: Control, PANEL C: PG, PANEL D: PG+MG1).

FIG. 22 PANEL A shows the quantification of C. albicans 2 stock inoculum. FIG. 22 PANELS B-E show the quantification of C. albicans 2 treated with different compositions (PANEL B: Control, PANEL C: PG, PANEL D: PG+1:10 MG1, PANEL E: PG+1:100 MG1).

FIG. 23 PANEL A shows the quantification of E. coli stock inoculum. FIG. 23 PANELS B-F show the quantification of E. coli treated with different compositions (PANEL B: Control, PANEL C: PG, PANEL D: PG+MG1, PANEL E: PG+1:10 MG1, PANEL F: PG+1:100 MG1).

FIG. 24 PANEL A shows the quantification of E. cloacae stock inoculum. FIG. 24 PANELS B-F show the quantification of E. cloacae treated with different compositions (PANEL B: Control, PANEL C: PG, PANEL D: PG+MG1, PANEL E: PG+1:10 MG1, PANEL F: PG+1:100 MG1).

FIG. 25 PANEL A shows the quantification of Klebsiella pneumoniae stock inoculum. FIG. 25 PANELS B-F show the quantification of Klebsiella pneumoniae treated with different compositions (PANEL B: Control, PANEL C: PG, PANEL D: PG+MG1, PANEL E: PG+1:10 MG1, PANEL F: PG+1:100 MG1).

FIG. 26 PANEL A shows the quantification of Burkholderia cepacia stock inoculum. FIG. 26 PANELS B-F show the quantification of Burkholderia cepacia treated with different compositions (PANEL B: Control, PANEL C: PG, PANEL D: PG+MG1, PANEL E: PG+1:10 MG1, PANEL F: PG+1:100 MG1).

FIG. 27 PANEL A shows the quantification of P. deurginosa 1 stock inoculum. FIG. 27 PANELS B-D show the quantification of P. aeurginosa 1 treated with different compositions (PANEL B: Control, PANEL C: PG, PANEL D: PG+MG1).

FIG. 28 PANEL A shows the quantification of P. deurginosa 2 stock inoculum. FIG. 28 PANELS B-E show the quantification of P. aeurginosa 2 treated with different compositions (PANEL B: Control, PANEL C: PG, PANEL D: PG+1:10 MG1, PANEL E: PG+1:100 MG1).

FIG. 29 PANEL A shows the quantification of Aspergillus niger stock inoculum. FIG. 29 PANELS B-F show the quantification of Aspergillus niger treated with different compositions (PANEL B: Control, PANEL C: PG, PANEL D: PG+MG1, PANEL E: PG+1:10 MG1, PANEL F: PG+1:100 MG1).

### DETAILED DESCRIPTION

Provided herein are antibacterial, antifungal, and antiviral formulations that are non-toxic, nonflammable, and biodegradable. The formulations provided herein can work against broad spectrum of drug sensitive and drug resistant pathogens and viruses. The formulations provided herein can be used for treatment of topical infections, environmental clean-up, and medical instrument disinfections.

### Pharmaceutical Compositions

The compositions of the disclosure can comprise, for example, a surfactant, a chelating agent, a pH modifier, and a solvent. The compositions of the disclosure can comprise a surfactant, for example, one or more alkanolamines, alkylarylsulfonates, amine oxides, poly(oxyalkylene) compounds (e.g., block co-polymers comprising alkylene oxide repeat units), carboxylated alcohol ethoxylates, ethoxylated alcohols, ethoxylated alkyl phenols, ethoxylated amines and amides, ethoxylated fatty acids, ethoxylated fatty esters and oils, fatty esters, fatty acid amides, glycerol esters, glycol esters, sorbitan esters, imidazoline derivatives, lechithin and lechithin derivatives, lignin and lignin derivatives, monoglycerides and monoglyceride derivatives, olefin sulfonates, phosphate esters and derivates, propoxylated and ethoxylated fatty acids or alcohols or alkyl phenols, sorbitan derivatives, sucrose esters and derivatives, sulfates or alcohols or ethoxylated alcohols or fatty esters, sulfates or sulfonates of dodecyl and tridecyl benzenes or condensed naphthalenes or petroleum, sodium dodecyl sulfate (SDS), sodium lauryl sulfate, sulfosuccinates and derivatives, or tridecyl and dodecyl benzene sulfonic acids. In some embodiments, the compositions of the disclosure can comprise more than one surfactant. In some embodiments, the surfactant can be a cationic surfactant, such as pH-dependent primary, secondary, or tertiary amines. In some embodiments, the surfactant can comprise ethyl lauroyl arginate or lauroyl lysine. In some embodiments, the cationic surfactant can comprise a quaternary ammonium compound, such as cetyltrimethylammonium bromide (CTAB), cetyltrimethylammonium chloride (CTAC), cetyltrimethylammonium fluoride (CTAF), cetyltrimethylammonium iodide (CTAI), benzylalkonium chloride (BAC), benzethonium chloride (BZT), methyl benzethonium chloride, dimethyldioctadecylammonium chloride, dioctadecyldimethylammonium bromide (DODAB), didecyl dimethyl ammonium chloride, dioctyl dimethyl ammonium chloride, alkyl dimethyl benzyl ammonium chloride, diisobutyl phenoxy ethoxy ethyl dimethyl benzyl ammonium chloride, alkyl dimethyl benzyl ammonium saccharinate, octyl decyl dimethyl ammonium chloride, alkyl dimethyl ethyl benzyl ammonium chloride, methyldodecylbenzyl ammonium chloride, methyldodecylxylene-bis-trimethyl ammonium chloride, cetyl pyrinidinium chloride, cetrimonium bromide and combinations thereof. A surfactant can damage cellular membranes of microorganisms (e.g., bacteria, fungi, enveloped viruses). At some concentrations, a surfactant can make lipid bilayers permeable. A cationic surfactant (e.g., CTAC or CTAB) can promote production of reactive oxygen species (ROS) inside the cell of the microorganism, which greatly enhances killing. In some embodiments, the compositions of the disclosure can comprise any combination of the surfactants provided herein.

The compositions of the disclosure can comprise a cationic surfactant at a concentration of about 0.2 mM to 50 mM, for example, about 0.2 mM, about 0.5 mM, about 1 mM, about 2 mM, about 5 mM, about 10 mM, about 20 mM, or about 50 mM. In some embodiments, the compositions of the disclosure can comprise a cationic surfactant (e.g., CTAC or CTAB) at a concentration of about 1 mM. In some embodiments, the compositions of the disclosure can comprise a cationic surfactant (e.g., CTAC or CTAB) at a concentration of about 2 mM. In some embodiments, the compositions of the disclosure can comprise a cationic surfactant (e.g., CTAC or CTAB) at a concentration of about 5 mM. In some embodiments, the compositions of the disclosure can comprise a cationic surfactant (e.g., CTAC or CTAB) at a concentration of about 10 mM.

The concentration of the surfactant in the composition can be from about 0.01 wt % to about 1 wt %. In some embodiments, the concentration of the surfactant can be from about 0.05 wt % to about 0.5 wt %. In some embodiments, the concentration of the chelating agent can be about 0.01 wt %, about 0.02 wt %, about 0.05 wt %, about 0.1 wt %, about 0.2 wt %, about 0.5 wt %, or about 1 wt %.

The compositions of the disclosure can comprise a chelating agent. The chelating agent, or chelator, can comprise one or more organic or inorganic compounds that contain two or more electron donor atoms that form coordinate bonds to metal ions or other charged particles. After the first such coordinate bond, each successive donor atom that binds creates a ring containing the metal or charged particle. The structural aspects of a chelating agent can comprise coordinate bonds between a metal or charged particle, which can serve as an electron acceptor, and two or more atoms in the molecule of the chelating agent, or ligand, which can serve as the electron donors. The chelating agent can be bidentate, tridentate, tetradentate, pentadentate, etc., according to the number of donor atoms capable of simultaneously complexing with the metal ion or charged particle.

The chelating agent can comprise an organic compound that contains a hydrocarbon linkage and two or more functional groups. The same or different functional groups can be used in a single chelating agent. The functional groups for chelation can comprise ═O, —OR, —NR₂, ═NR, ═NOR, and/or ═N—R*—OR, wherein R is H or alkyl; and R* is alkylene. The functional groups can comprise phosphate and/or phosphonate groups. The alkyl groups can contain from 1 to 10 carbon atoms, and in some embodiments from 1 to 4 carbon atoms. The alkylene groups may can contain from 2 to 10 carbon atoms, and in some embodiments from 2 to 4 carbon atoms. In some embodiments, the chelating agent can comprise one or more of ethylenediaminetetraacetic acid (EDTA), diethylenetriaminepentaacetic acid (DTPA), N,N-Dicarboxymethyl glutamic acid (GLDA), 2,3-dimercaptopropane 1-sulfonate (DMPS), Prussian blue, glutathione, dimercaptosuccinic acid (succimer, DMSA), deferoxamine (DFO), triethylenetetramine (TETA), desferrioxamine (DFOA), citric acid, peptides, amino acids including short chain amino acids, aminopolycarboxylic acids, gluconic acid, glucoheptonic acid, organophosphonates, bisphosphonates (e.g., pamidronate), or an inorganic polyphosphate.

Salts of one or more of the foregoing chelating agents can be used. Non-limiting examples of salts include sodium, calcium, and zinc salts of the foregoing. A sodium, calcium, and/or zinc salts of DTPA can be used. Salts of the foregoing chelating agents can be formed when neutralizing the agent with, for example, sodium hydroxide. Mixtures of two or more of any of the foregoing can be used.

In some embodiments, the chelating agent can comprise GLDA or a pharmaceutically-acceptable salt thereof. GLDA is a chelating agent that can be made from natural, biodegradable, renewable raw materials. GLDA is readily biodegradable with a high level of solubility over a wide pH range and is a greener alternative to many other chelating agents. In some embodiments, a biodegradable substance can erode or degrade, for example, in vivo to form smaller chemical fragments. Degradation can occur, for example, by enzymatic, chemical, or physical processes. Due to the degradability of GLDA or a salt thereof, the formulation comprising such substance can be used on living organisms.

Salts, for example, pharmaceutically-acceptable salts can comprise metal salts. In some embodiments, a metal salt can comprise a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

In some embodiments, pharmaceutically-acceptable salts can comprise ammonium salts. In some embodiments, an ammonium salt is a triethyl amine salt, potassium iodate, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

The chelating agent (e.g., GLDA or DTPA) can enter the cells of bacteria or fungi and bind divalent cations, such as Ca, Mg, Mn, Zn, Cu, and Fe, which are critical to viability (e.g., signal transduction, RNA/DNA integrity, energy production, or detoxification). Therefore, the chelating agent can promote the killing of the microorganisms.

The compositions of the disclosure can comprise a chelating agent at a concentration of about 1 mM to 200 mM, for example, about 1 mM, about 2 mM, about 5 mM, about 10 mM, about 15 mM, about 20 mM, about 30 mM, about 40 mM, about 50 mM, about 60 mM, about 70 mM, about 80 mM, about 90 mM, about 100 mM, about 120 mM, about 150 mM, or about 200 mM. In some embodiments, the compositions of the disclosure can comprise a chelating agent (e.g., GLDA or a salt thereof) at a concentration of about 5 mM. In some embodiments, the compositions of the disclosure can comprise a chelating agent (e.g., GLDA or a salt thereof) at a concentration of about 10 mM. In some embodiments, the compositions of the disclosure can comprise a chelating agent (e.g., GLDA or a salt thereof) at a concentration of about 20 mM. In some embodiments, the compositions of the disclosure can comprise a chelating agent (e.g., GLDA or a salt thereof) at a concentration of about 30 mM. In some embodiments, the compositions of the disclosure can comprise a chelating agent (e.g., GLDA or a salt thereof) at a concentration of about 50 mM.

The concentration of the chelating agent in the composition can be from about 0.1 wt % to about 5 wt %. In some embodiments, the concentration of the chelating agent can be from about 0.5 wt % to about 2 wt %. In some embodiments, the concentration of the chelating agent can be about 0.1 wt %, about 0.2 wt %, about 0.5 wt %, about 1 wt %, about 1.5 wt %, about 2 wt %, or about 5 wt %.

The compositions of the disclosure can comprise a pH modifier. The pH modifier can be used to adjust the pH of the compositions. Maintaining pH stability can be important for certain compositions. The pH modifier can comprise any suitable acids and/or bases. Non-limiting examples of acids comprise hydroxyacetic (glycolic) acid, citric acid, formic acid, acetic acid, propionic acid, butyric acid, valeric acid, caproic acid, trichloroacetic acid, urea hydrochloride, benzoic acid, oxalic acid, malonic acid, gluconic acid, itaconic acid, succinic acid, glutaric acid, maleic acid, fumaric acid, lactic acid, adipic acid, terephthalic acid, malic acid, succinic acid, tartaric acid, sulfuric acid, sulfamic acid, methylsulfamic acid, hydrochloric acid, hydrobromic acid, nitric acid, or a mixture thereof. Non-limiting examples of bases comprise ammonia, ammonium hydroxide, amines, alkanolamines, amino alcohols, borates, carbonates, hydroxides, silicates, or a mixture thereof.

The compositions of the disclosure can comprise a pH modifier at a concentration of about 1 mM to about 150 mM, for example, about 1 mM, about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 120 mM, or about 150 mM. In some embodiments, the compositions of the disclosure can comprise a pH modifier at a concentration of about 5 mM. In some embodiments, the compositions of the disclosure can comprise a pH modifier at a concentration of about 10 mM. In some embodiments, the compositions of the disclosure can comprise a pH modifier at a concentration of about 15 mM. In some embodiments, the compositions of the disclosure can comprise a pH modifier at a concentration of about 20 mM. In some embodiments, the compositions of the disclosure can comprise a pH modifier at a concentration of about 30 mM. In some embodiments, the pH modifier can adjust the pH of the composition to a value from about 5 to about 8.5, e.g., about 5, about 5.4, about 6, about 6.2, about 6.5, about 6.7, about 7, about 7.4, about 7.2, about 7.5, about 7.7, 8.0 or about 8.5.

The compositions of the disclosure can comprise a solvent. In some embodiments, the solvent can comprise water. In some embodiments, the solvent can be substantially water. In some embodiments, the compositions of the disclosure can comprise a mixture of solvents, for example, a mixture of water and an organic solvent. The compositions of the disclosure can comprise, for example, about 25%, about 50%, about 75%, or about 100% of an organic solvent. Non-limiting examples of organic solvent comprise propylene glycol (PG), dimethyl sulfoxide (DMSO), Ficoll® 400 (e.g., 7.5% w/v final), low molecular weight methylcellulose, methanol, ethanol, polyvinyl alcohol, isocetyl alchol, oleyl alcohol, propanol, polyethylene glycol, glycerol, aloe vera extract, paraffin oil, petroleum gel, petroleum jelly, acetylated lanolin alcohol, almond oil, apricot kernel oil, avocado oil, cocoa butter, coconut butter, corn oil, cotton seed oil, evening primrose oil, hydrogenated vegetable oil, isodecyl oleate, jojoba oil, olive oil, peanut oil, PEG 8 castor oil, sandalwood seed oil, sesame oil, shark liver oil, soybean oil, or sulfated jojoba oil.

The compositions of the disclosure can comprise a solvent (e.g., water) at a percentage of at least 95 percent by weight (wt %), at least 95.5 wt %, at least 96 wt %, at least 96.5 wt %, at least 97 wt %, at least 97.5 wt %, at least 98 wt %, at least 98.5 wt %, or more.

The compositions of the disclosure can also comprise additional non-comodogenic or comodogenic ingredients, for example, colloidal silicon dioxide, beeswax, butyl stearate, capric acid, caprylic acid, carnuba wax, ceteareth 20, cetyl acetate, cetyl alcohol, cetearyl alcohol, D&C red #17, D&C red #19, D&C red #21, D&C red #27, D&C red #3, D&C red #30, D&C red #33, D&C red #36, D&C red #4, D&C red #40, D&C red #9, decyl oleate, di(2-ethylhexyl) succinate, dimethicone, dioctyl malate, diocyl succinate, eicosanoic acid, ethylhexyl palmitate, ethylhexyl pelargonate, glyceryl tricapylo/caprate, glyceryl stearate NSE, glyceryl stearate SE, glyceryl-3-diisostearate, hexylene glycol, isocetyl alcohol, isodecyl oleate, isopropyl isostearate, isopropyl linolate, isopropyl myristate, isopropyl palmitate, isostearyl isostearate, isostearyl neopentanoate, lanolin wax, laureth-23, laureth-4, lauric acid, mink oil, myristic acid, myristyl lactate, myristyl myristate, myristyl alcohol, oleth-10, oleth-20, oleth-3, oleth-3 phosphate, oleth-5, oleyl alcohol, palmitic acid, PEG 100 distearate, PEG 150 distearate, PEG 16 lanolin (Solulan 16), PEG 20 stearate, PEG 200 dilaurate, PEG 8 castor oil, PEG 8 stearate, PG caprylate/caprate, PG dicaprylate/caprate, PG dipelargonate, PG monostearate, phytantriol, polyglyceryl-3-diisostearate, PPG 10 cetyl ether, PPG 2 myristyl propionate, PPG 5 ceteth 10 phosphate, sorbitan isostearate, sorbitan oleate, squalene, steareth-10, stearateh-2, steareth-20, stearic acid, stearic acid TEA, stearyl alcohol, stearyl heptanoate, triethanolamine, water-soluble sulfur, xylene, or zinc dioxide.

The compositions of the disclosure can further comprise a dye, pigment, or colorant. The compositions of the disclosure can comprise an organic dye, pigment, or colorant; or an inorganic dye, pigment, or colorant. For example, a formulation of the disclosure can comprise a dye, such as Alcian yellow GXS (Ingrain yellow 1), Alizarin (Mordant red 11), Alizarin red S (Mordant red 3), Alizarin yellow GG (Mordant yellow 1), Alizarin yellow R (Mordant orange 1), Azophloxin (Acid red 1), Bismarck brown R (Vesuvine brown), Bismarck brown Y (Vesuvine phenylene brown), Brilliant cresyl blue (Cresyl blue BBS), Chrysoidine R (Basic orange 1), Chrysoidine Y (Basic orange 2), Congo red (Direct red 28), Crystal violet (Basic violet 3), Fuschsin acid (Acid violet 19), Gentian violet (Basic violet 1), Janus green, Lissamine fast yellow (Acid yellow 17), Malachite green, Martius yellow (Acid yellow 24), Meldola blue (phenylene blue), Metanil yellow (Acid yellow 36), Methyl orange (Acid orange 52), Methyl red (Acid red 2), Naphthalene black 12B (Amido black 10B or Acid black 1), Naphthol green B (Acid green 1), Naphthol yellow S (Acid yellow 1), Orange G (Acid orange 10), Purpurin (Verantin), Rose Bengal (Acid red 94), Sudan II (Solvent orange 7), Titan yellow (Direct yellow 9), Tropaeolin O (sulpho orange or acid orange 6), Tropaeolin OO (Acid orange 5), Tropaeolin OOO (Acid orange 7), Victoria blue 4R (Basic blue 8), Victoria blue B (Basic blue 26), Victoria blue R (Basic blue 11), or Xylene cyanol FF (Acid blue 147).

The compositions of the disclosure can further comprise viscosity modulators, such as a thickening agent. For example, the compositions of the disclosure can comprise a thickening agent, such as a starch (e.g., arrow root, cornstarch, katakuri starch, potato starch, sago, tapioca, cellulose), vegetable gum (alginin, guar gum, locust bean gum, xanthan gum), a protein (e.g., collagen, egg whites, gelatin), a sugar (e.g., agar, carrageenan), or sodium pytophosphate. The compositions of the disclosure can further comprise synthetic polymers e.g., thermogels (e.g., Pluronic-F127), polyvinyl alcohols, and methacrylates. In some embodiments, natural or synthetic polymers are added to a composition described herein to produce a viscous composition, e.g., a slurry. In some embodiments, the slurry permits application on substrates that are vertical and have uneven, permeable or impermeable, smooth or rough, non-porous or porous, or cracked surfaces. In some embodiments, the slurry, provides increased length of protection when applied in a surface. Non-limiting examples of such surfaces and materials include glass, metal, wood, plastic, cloth, tile, concrete, rock, leather, synthetic leather, plant seeds, eggs, door knobs, surgical tools and equipment, and animal/human skin (e.g., hairy, feathery, or not) and cavities (e.g., ears, nose, or vagina) or messy wounds.

In some embodiments, a slurry can be applied in a surface and allowed to dry. Once dried, the resulting film can subsequently be removed by peeling off. In some embodiments, the formulation diffuses out of the slurry and into the surroundings, leaving the surface sterile. While drying, the film absorbs some biological material such as pathogens. The biological materials thus absorbed are preserved and rendered non-infective. The biological materials thus absorbed can be subjected to forensic analysis by extracting out of the film DNA/proteins and subjecting to either DNA (PCR, RFLP etc.) or antigen (Western blot, ELISA, etc.) analysis.

In some embodiments, the compositions can comprise a polymer. In some embodiments, the polymer can comprise a naturally occurring polymer, e.g., cellulose, a polysaccharide, an enzyme, a protein, a polypeptide, or a nucleic acid. In some embodiments, the polymer can comprise a synthetic polymer, e.g., a polyvinyl alcohol, poly(meth)acrylic acid, polystyrene, polyolefin, or co-polymers. In some embodiments, the polymer can comprise a water-soluble film forming polymer. In some embodiments, the polymer can form a film upon drying. In some embodiments, the compositions can be substantially free of a water-soluble polymer. In some embodiments, the compositions can be substantially free of a water-soluble film forming polymer.

The compositions of the disclosure can further comprise stabilizers. For example, the compositions of the disclosure can comprise calcium-zinc, organo-calcium, lead, and tin-based stabilizers. The compositions of the disclosure can also comprise liquid and light stabilizers, such as hindered amine light stabilizers (HALS), benzophenone, or benzotriazole. Examples of stabilizers also include tris(2,4-di-tert-butylphenyl) phosphite, antioxidants (e.g., oxygen scavengers, such as phosphite esters; persistent radical scavengers, such as butylated hydroxytoluene; antiozonants; sequestrants; ultraviolet stabilizers). The compositions of the disclosure can include gelling agents, such as alginic acid, sodium alginate, potassium alginate, ammonium alginate, calcium alginate, agar, carrageenan, locust bean gum, pectin, or gelatin. In some embodiments, a thickening agent, such as polyethylene glycol, synthetic carbomers, petroleum jelly, paraffin, and wax can be used in the formulations of the disclosure.

The compositions of the disclosure can further comprise local anesthetics. For example, the compositions of the disclosure can comprise a local anesthetic, such as lignocaine (lidocaine, Remicaine®, Petercaine®), bupivacaine (e.g., Macaine®), ropivacaine (e.g., Naropin®), prilocaine, amethocaine (e.g., Tetracaine®), procaine, cinchocaine (e.g., Dibucaine®), mepivacaine (e.g., Carbocaine®), or etidocaine.

The compositions of the disclosure can further comprise tissue permeabilizers. The compositions of the disclosure can comprise a tissue permeabilizer, such as an organic solvent (e.g., methanol, acetone) or a detergent (e.g., saponin, Triton™ X-100, Tween®-20, natural terpenes).

The compositions of the disclosure can further comprise bleach activators. The compositions of the disclosure can comprise a bleach activator such as Tetraacetylethylenediamine (TAED), sodium nonanoyloxybenzenesulfonate (NOBS), tetraacetyl glycoluril, nitriles (e.g., cyanopyridine, cyanamides, cyanomorpholin, e.g., cyanomethyl trialkyl/arylammonium salts), or phthalimidoperoxyhexanoic acid (PAP). In some embodiments, the formulations provided herein can generate intracellular reactive oxygen species (ROS) in an organism. In some embodiments, a formulation comprising a bleach activator can generate an increased amount of intracellular (ROS) in an organism compared to a formulation not comprising a bleach activator.

The compositions of the disclosure can further comprise skin softeners. The compositions of the disclosure can comprise a skin softener, such as lentil seed extract, hexyl laurate, hexyldecanol, hydrogenated polyisobutene, hydrolyzed glycosaminoglycans, hydrolyzed jojoba esters, isoamyl laurate, Limnanthes alba, lyceum barbarum fruit extract, methyl gluceth-20, methyl glucose sesquistearate, methylglucoside phosphate, millet seed extract, neopentyl glycol diheptanoate, octyldodecyl myristate, octyldodecyl neopentanoate, oleyl erucate, Oryza sativa cera, polyglyceryl-10 laurate, tridecyl trimellitate, trimethylsiloxysilicate, trioctyldodecyl citrate, triticum volgare (wheat) germ extract, Triticum vulgare oil, whey protein, xymenynic acid, Cocos nucifera (coconut) fruit extract, caprylyl caprylate/caprate, ethylhexyl olivate, urea, or tocopheryl acetate.

The compositions of the disclosure can further comprise an exfoliant. The compositions of the disclosure can comprise an exfoliant, such as betaine salicylate, beta hydroxy acid, bromelain, alpha hydroxy acid, ammonium glycolate, amygdalic acid, ananas sativus fruit extract, azelaic acid, gluconolactone, lactic acid, lactobionic acid, glycolic acid, malic acid, mandelic acid, papain, papaya extract, polyhydroxy acid, salicylic acid, tartaric acid, or urea.

The compositions of the disclosure can further comprise amino acids, peptides, proteins, or proteases. In some embodiments, the compositions of the disclosure can comprise poly-L-lysine, arginine, bacitracin, milk hydrolysate, alcalase, collagenase, or keratinase.

The compositions of the disclosure can further comprise an antibiotic. The compositions of the disclosure can comprise antibiotics, such as penicillins, cephalosporins, macrolides, fluoroquinolones, sulfonamides, tetracyclines, bacitracin, cycloserine, polymyxin B, polymyxin E (Colistin), or aminoglycosides. For example, a formulation of the disclosure can further comprise a penicillin, such as penicillin or amoxicillin. A formulation of the disclosure can further comprise a cephalosporin, such as cephalexin. A formulation of the disclosure can further comprise a macrolide, such as erythromycin, clarithromycin, or azithromycin. A formulation of the disclosure can further comprise a fluoroquinolone, such as ciprofloxacin, levofloxacin, or ofloxacin. A formulation of the disclosure can further comprise a sulfonamide, such as co-trimoxazole or trimethoprim. A formulation of the disclosure can further comprise a tetracycline, such as tetracycline (sumycin, panmycin) or doxycycline. A formulation of the disclosure can further comprise an aminoglycoside, such as gentamicin or tobramycin.

A composition of the disclosure can be substantially free of heavy metals. A composition of the disclosure can be substantially free of enzymes.

A composition of the disclosure can be used, for example, before, during, or after treatment of a subject with a second pharmaceutical agent. In some embodiments, when the composition is used in combination with a second pharmaceutical agent, the composition and the agent show a synergistic effect. In some embodiments, the second pharmaceutical agent is selected from triclosan, chlorhexidine, or mupirocin.

In some embodiments, a composition of the disclosure can be used in combination with a second pharmaceutical agent on a subject before surgery. For example, the composition of this disclosure can be used in combination with Mupirocin for pre-surgery nasal decolonization.

A composition of the disclosure can be a combination of any pharmaceutical compounds described herein with other chemical components, such as carriers, stabilizers, diluents, dispersing agents, suspending agents, thickening agents, fragrants, odorants, deodorants, and/or excipients. The pharmaceutical composition facilitates administration of the compound to an organism. Pharmaceutical compositions can be administered in therapeutically-effective amounts as pharmaceutical compositions by various forms and routes including, for example, ophthalmic, nasal, vaginal, and topical administration. In some embodiments, the compositions of the disclosure further comprise certain additives that improve the manufacturing process or lend other desirable qualities to the final composition. In some embodiments, when the composition is used in combination with a second pharmaceutical compound, the composition and the second pharmaceutical compound show synergistic effect. In some embodiments, the second pharmaceutical compound is selected from a chelating agent (e.g., triethanolamine), a pro-oxidant (e.g., KIO₃, ascorbic acid), and a distractant (e.g., n-acetylcysteine). In some embodiments, the second pharmaceutical agent is a chelating agent, e.g., triethanolamine.

### Applications

The compositions of the disclosure can be used to clean or disinfect infected surfaces, such as wood, metal, glass, concrete, painted surfaces, or plastic surfaces. The compositions of the disclosure can be applied to a non-infected surface to reduce a likelihood that a pathogen forms on the surface (e.g., a protectant). The formulation of the disclosure can be used to clean, disinfect, or protect a surface made of or comprising a porous or non-porous material.

In some embodiments, the surface can comprise horizontally or vertically aligned non-porous substrates, such as floors and walls, counter tops, table tops, medical equipment, gurneys, heart stress test room surfaces, toilets, or seats. In some embodiments, the formulations of the disclosure can be used to protect, clean or disinfect complex, three-dimensional structures, such as faucets, tools, or other equipment. In some embodiments, the formulations of the disclosure can be used to protect, clean, or disinfect, or decontaminate vehicle interiors, buildings, medical facilities (e.g., hospitals, dental clinics), articles of manufacture, buildings and infrastructure intended for demolition, military assets, airplanes, military ships, or civilian ships. In some embodiments, the formulations of the disclosure can be used to protect, disinfect, or decontaminate food (e.g., eggs, fruits, or vegetables) or packaged items. In some embodiments, the formulations of the disclosure can be used in combination with other products, for example, cleaning products, shampoos, or soaps. In some embodiments, the formulations of the disclosure can be used for wound management, or topical treatment. The formulations of the disclosure can be used to treat human skin, animal skin, wounds, and body cavities, for example, the mouth, vagina, ears, or nose. In some embodiments, the formulations of the disclosure can be used to disinfect medical and/or surgical equipment. In some embodiments, the pharmaceutical composition of the disclosure can be used to treat an infection resistant to a second pharmaceutical agent, e.g., triclosan, or chlorhexidine. In some embodiments, the formulations of the disclosure can be used to lessen the likelihood of contracting nosocomial infections. In some embodiments, the formulations of the disclosure can be used to treat nosocomial infections.

Nosocomial infections are infections acquired during the process of receiving health care that was not present during the time of admission. Infection occurs when pathogen(s) spread to a susceptible subject. Nosocomial infections can occur in different areas of healthcare facilities, e.g., hospitals, long-term care facilities, and ambulatory settings. Nosocomial infections can also appear after discharge. The emergence of multidrug-resistant organisms is another complication seen with Nosocomial infections. Robust disinfecting/cleaning compositions with broad spectrum, multidrug-resistant organism killing activity, low toxicity, and biodegradability can be used to lessen the likelihood of contracting and treat nosocomial infections.

In some embodiments, the formulations provided herein can have full kill of broad spectrum and multidrug-resistant microorganism in less than 10 minutes, less than 5 minutes, or less than 2 minutes. In some embodiments, the formulations provided herein can have sustained killing activity. In some embodiments, the formulations provided herein can disinfect a surface or object for about 2 minutes to 4 weeks or longer. In some embodiments, bacterial or fungal growth does not appear in a disinfected surface for at least 2 minutes to 28 days or longer after treatment with the formulations provided herein.

The formulations provided herein can be used to treat fungal infections. For example, the formulations of the disclosure can treat Candida auris (C. auris), cryptococcal meningitis, fungal eye infections, oral candidiasis, vaginal candidiasis, candida vaginitis, tinea pedis (athlete's foot), pulmonary apergillosis, chronic pulmonary apergillosis, toe and hand nails infected with Trichophyton rubrum, tinia capitis (tinea tonsurans), ringworm, mucormycosis, or blastomycosis. The formulations of the present disclosure can also be used to treat infections caused by an Ascomycetous fungus (e.g., pezizomycotina, saccharomycotina, and taphrinomycotina), Trichophyton, epidermophyton, microsporum. Aspergillus, Blastomyces, Cryptococcus neoformans, Cryptococcus gattii, Trichophyton rubrum, Aspergillus niger, stachybotrys, Batrachochytrium dendrobatidis (frog Bd fungus), or Blastomyces dermatitidis.

C. auris is a yeast-like fungus related to Candida albicans. C. auris is one of the few species of the Candida genus that can cause candidiasis in humans. Candidiasis can be acquired in hospitals by patients with weakened immune systems. C. auris can cause invasive candidiasis in which the bloodstream (fungemia), the central nervous system, and internal organs are affected. The fungus causes invasive infections with a high death rate (about 57%), and causes mainly bloodstream, wound, and ear infections. C. auris is invasive and multidrug-resistant in contrast to other fungal diseases and is usually associated with outbreaks in healthcare settings like hospitals.

Meningitis, an infection of the lining of the spinal cord and brain, is the most common illness caused by Cryptococcus. Cryptococcal meningitis can cause coma and death, and can also infect the skin, lungs, or other parts of the body. The risk of cryptococcal infection is highest when a person's CD4 counts are below 100. Cryptococcal meningitis is a major HIV-related opportunistic infection.

Fungal eye infections are rare but can be very serious. The most common way for someone to develop a fungal eye infection is from an eye injury, particularly if the injury was caused by plant material, such as a stick or a thorn. Inflammation or infection of the cornea is known as keratitis, and inflammation or infection of the interior of the eye is called endophthalmitis. In some embodiments, fungal eye infections are caused by Moniliaceae, Dematiaceae, and yeasts. Moniliaceae includes non-pigmented filamentary fungi, such as Fusarium and Aspergillus species; Dematiaceae includes pigmented filamentary fungi, such as Curvularia and Lasiodiplodia species; and yeasts include Candida species.

Cryptococcus neoformans and Cryptococcus gattii are species of fungus that can cause crytococcosis, also known as cryptococcal disease. Cryptococcus is a potentially fatal fungal disease that can be acquired by inhalation of the infections propagule from the environment. Cryptococcus does not spread from person to person.

Batrachochytrium dendrobatidis is a species of fungus that can cause chytridiomycosis in amphibians such as frogs. Batrachochytrium dendrobatidis can be introduced to amphibians through water exposure and can eventually lead to cardiac arrest and death.

A formulation of the disclosure can be used as a treatment against bacterial agents. Non-limiting examples of bacterial agents that can be treated with a formulation of the disclosure include Bacillus anthracis (anthrax), Franscisella tularensis (tularemia), Yersinia pestis (plague), Clostridium botulinum (botulism), Coxiella burnetti (Q fever), Brucella species (brucellosis), Burkholderia mallei (glanders), Clostridium perfringens (clostridial necrotizing enteritis), Vibrio cholerae (cholera), and Porphyromonas gingivalis (gingavitis, and can cause Alzheimer's Disease).

A formulation of the disclosure can be used as a treatment against viral agents. Non-limiting examples of viral agents that can be treated with a formulation of the disclosure include, for example, arenaviruses such as Lassa virus, Junin mammarenavirus, and Machupo virus; filoviruses such as Ebola virus, and Marburg virus; Variola major (smallpox); Nipah virus; Hantaviruses; Nairovirus (Bunyaviradae family); lentiviruses such as HIV; varicella-zoster virus; paramyxovirus; rubeola virus; rhinovirus; coronavirus such as SARS-COV; respiratory syncytial virus; influenza virus; noroviruses; hepatitis A; rotavirus; astrovirus; and Norwalk-like viruses.

In some embodiments, the pharmaceutical composition can be used to treat an infection by a microorganism, e.g., a bacterial infection, a fungal infection, a viral infection. In some embodiments, the infection can be caused by a spore. In some embodiments, the infection is a bacterial infection. In some embodiments, the infection is a nosocomial infection. In some embodiments, the infection is an opportunistic infection. In some embodiments, the spore can be a spore of a yeast, e.g., Aspergillus niger.

In some embodiments, the pharmaceutical composition can be used to treat a bacterial infection caused by a bacteria selected from Listeria meningitides, Staphylococcus aureus, Bacillus cereus, Clostridium sporogenes, Bacillus subtilis, Mycobacterium smegmatis, Streptococcus Pyogenes, Enterococci, Mycobacterium smegmatis, Pseudomonas aeruginosa, Burkholderia cepacia, Burkholderia thailandesis, Acinetobacter baumannii, Escherichia coli, Proteus mirabilis, Salmonella infantis, Yesenia pestis, Klebsiella pneumoniae, Stenotrophomonas maltophilia, Carbapenem-resistant Enterobacteriaceae, or other types of bacteria thereof. In some embodiments, the bacterial infection can be caused by gram-positive bacteria, e.g., Listeria meningitides, Staphylococcus aureus, Bacillus cereus, Clostridium sporogenes, Bacillus subtilis, Mycobacterium smegmatis, Streptococcus Pyogenes, Enterococci, Mycobacterium smegmatis. In some embodiments, the bacterial infection by Staphylococcus can be methicillin-resistant. In some embodiments, the bacterial infection by Enterococci can be vancomycin-resistant. In some embodiments, the bacterial infection can be caused by gram-negative bacteria, e.g., Pseudomonoas aeruginosa, Burkholderia cepacia, Burkholderia thailandesis, Acinetobacter baumannii, Escherichia coli, Proteus mirabilis, Salmonella infantis, Yesenia pestis, Klebsiella pneumoniae, Stenotrophomonas maltophilia, Carbapenem-resistant enterobacteriaceae. In some embodiments, the bacterial infection can be caused by colistin resistant bacteria. In some embodiments, the bacterial infection (e.g., Stenotrophomonas maltophilia) can be polymyxin E (PME) resistant. In some embodiments, the bacterial infection can be multidrug-resistant. In some embodiments, the bacterial infection can be caused by spore forming bacteria. In some embodiments, the bacterial infection can be caused by non-spore forming bacteria. In some embodiments, the bacterial infection can be caused by aerobic bacteria, e.g., Burkholderia thailandesis. In some embodiments, the bacterial infection can be caused by anaerobic bacteria, e.g., Listeria Meningitides. In some embodiments, the bacterial infection can be flesh-eating bacteria (e.g., Streptococcus Pyogenes). In some embodiments, the bacterial infection can be contagious. In some embodiments, the bacterial infection can be transmitted via feces. In some embodiments, the bacterial infection (e.g., from Proteus mirabilis) can lead to urinary tract infections. In some embodiments, the bacterial infection (e.g., from Escherichia coli) can lead to food poisoning. In some embodiments, the bacterial infection can lead to respiratory system infections. In some embodiments, the bacterial infection is a respiratory pathogen. In some embodiments, the bacterial infection can lead to dermatitis. In some embodiments, the bacterial infection can lead to soft tissue infections. In some embodiments, the bacterial infection can lead to bacteremia. In some embodiments, the bacterial infection can lead to bone and joint infections. In some embodiments, the bacterial infection can lead to gastrointestinal infections. In some embodiments, the bacterial infection can lead to systemic infections (e.g., sarcoidosis, neoplasm, serositis, metastatic carcinoma, rheumatoid arthritis, system lupus erythematosus). In some embodiments, the bacterial infection can occur in people with weakened immune systems (e.g., organ transplant patients, cancer patients, hospitalized patients, stem cell transplant patients, people living with HIV/AIDS). In some embodiments, the bacterial infection can occur in people who are otherwise healthy. In some embodiments, the bacterial infection is a secondary infection. In some embodiments, the subject having the infection has a disease. In some embodiments, the subject having the infection has AIDS. In some embodiments, the subject having the infection is immunocompromised. In some embodiments, the bacterial infection is a nosocomial infection. In some embodiments, the bacterial infection is a secondary bacterial infection. In some embodiments, the subject having the secondary bacterial infection has a primary infection, e.g., a bacterial infection, a fungal infection, a viral infection. In some embodiments, the secondary infection is caused by a drug resistant bacterial strain, e.g., multiple drug resistant bacterial stain, e.g., antibiotic resistant bacterial strain.

In some embodiments, the pharmaceutical composition can be used to treat a fungal infection caused by a fungus (e.g., yeast, mold). In some embodiments, the pharmaceutical composition can be used to treat a fungal infection caused by a fungus selected from Cryptococcus neoformans, Candida auris, Candida albicans, Candida parapsilosis, Candida lusitaniae, Candida glabrata, Candida krusei, Candida duobushaemulonii, Kodameae ohmeri, Candida haemuloni, Aspergillus niger, Botrytis cinerea or other types of fungus thereof. In some embodiments, the infection can be a fungal infection. In some embodiments, the infection can be caused by fungus that can grow as yeast, e.g., Cryptococcus neoformans, Candida auris, Candida albicans, Candida parapsilosis, Candida lusitaniae, Candida glabrata, Candida krusei, Candida duobushaemulonii, Kodamede ohmeri, Candida haemuloni. In some embodiments, the fungal infection can be caused by fungal mold, e.g., Aspergillus niger, Botrytis cinerea. In some embodiments, the fungal infection can be caused by a unicellular type of fungus. In some embodiments, the fungal infection can be caused by fungus that can grow as multicellular filaments. In some embodiments, the fungal infection can be a respiratory pathogen. In some embodiments, the fungal infection can be opportunistic infections. In some embodiments, the fungal infection can occur in people with weakened immune systems (e.g., organ transplant patients, cancer patients, hospitalized patients, stem cell transplant patients, people living with HIV/AIDS). In some embodiments, the fungal infection can occur in people who are otherwise healthy. In some embodiments, the fungal infection is a nosocomial infection. In some embodiments, the fungal infection is a secondary fungal infection. In some embodiments, the subject having the secondary fungal infection has a primary infection, e.g., a bacterial infection, a fungal infection, a viral infection. In some embodiments, the secondary infection is caused by a drug resistant fungal strain, e.g., multiple drug resistant fungal stain, e.g., antibiotic resistant fungal strain. In some embodiments, the pharmaceutical composition can be used to treat a viral infection. In some embodiments, the viral infection can be caused by a positive-sense single-stranded RNA virus (e.g., Bacteriophage MS2). In some embodiments, the viral infection can be caused by enveloped human viruses (e.g., Sars-COV-2). In some embodiments, the viral infection can occur in people with weakened immune systems (e.g., organ transplant patients, cancer patients, hospitalized patients, stem cell transplant patients, people living with HIV/AIDS). In some embodiments, the viral infection can occur in people who are otherwise healthy. In some embodiments, the viral infection can be caused by arenaviruses such as Lassa virus, Junin mammarenavirus, and Machupo virus; filoviruses such as Ebola virus, and Marburg virus; Variola major (smallpox); Nipah virus; Hantaviruses; Nairovirus (Bunyaviradae family); lentiviruses such as HIV; varicella-zoster virus; paramyxovirus; rubeola virus; rhinovirus; coronavirus; respiratory syncytial virus; influenza virus; noroviruses; hepatitis A; rotavirus; astrovirus; and Norwalk-like viruses. In some embodiments, the viral infection is a nosocomial infection. In some embodiments, the viral infection is a secondary fungal infection. In some embodiments, the subject having the secondary viral infection has a primary infection, e.g., a bacterial infection, a fungal infection, a viral infection. In some embodiments, the secondary infection is caused by a drug resistant viral strain, e.g., multiple drug resistant viral stain, e.g., antibiotic resistant viral strain.

### Methods of Use

In an aspect, the present disclosure provides a method of killing a microorganism. The method comprises administering to the microorganism an effective amount of a composition provided herein in a mixture. The microorganism can comprise a bacterium, a fungus (e.g., a mold), a yeast, a spore, or a virus.

In another aspect, the present disclosure provides a method of cleaning or disinfecting a surface or an object. The method comprises contacting the surface or the object with an effective amount of a composition provided herein in a mixture. The surface or the object can be infected by a bacterium, a fungus (e.g., a mold), a yeast, a spore, or a virus.

The contacting can be performed by any suitable way of applying the composition. In some embodiments, the composition can be used to impregnate a suitable material, e.g., wipes or masks. In some embodiments, the composition can be used to impregnate a face mask to prepare an antibacterial, antifungal and antiviral protector face mask. Non-limiting examples of contacting include pouring, spraying, mopping, and wiping (e.g., wipes including hand-wipes). In some embodiments, the contacting can be performed at a temperature from about −80° C. to about 150° C.

In some embodiments, the present disclosure provides a method of treating an infection, e.g., primary infection or a secondary infection, in a subject. The method comprises administering to the subject a therapeutically-effective amount of a pharmaceutical composition provided herein in a unit dosage form. The infection can be caused by a bacterium, a fungus (e.g., a mold), a yeast, a spore, or a virus.

In some embodiments, the formulations and the method provided herein can kill bacteria and fungi (including fungal spores), e.g., multi-drug resistant bacteria and fungi (including fungal spores) across a very wide spectrum of pH. In some embodiments, the formulations provided herein can be from about 5 to about 8.5, e.g., about 5, about 5.4, about 6, about 6.2, about 6.5, about 6.7, about 7, about 7.4, about 7.2, about 7.5, about 7.7, 8.0 or about 8.5.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and/or preservatives.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and/or preservatives.

For cleaning/disinfecting application, the compositions can be formulated into a variety of compositions, such as solutions, suspensions, or aerosols.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the include liquid, powder, gel, aerosol, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the disclosure is administered in combination with, before, or after an antibiotic. If simultaneously, the multiple therapeutic agents can be provided in a single, unified form, or in multiple forms, for example, as multiple separate topical treatments. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged topicals, vials, or ampoules. Aqueous suspension compositions can be packaged in single-dose non-reclosable containers. Multiple-dose reclosable containers can be used, for example, in combination with or without a preservative. Formulations for administration can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid, aerosols, suspensions, or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, fragrances, coating agents, plasticizers, preservatives, suspending agents, emulsifying agents, plant cellulosic material and spheronization agents, and any combination thereof.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

In some embodiments, the pharmaceutical compositions of the disclosure can be formulated as leave-on sprays for use to treat mold. In some embodiments, the pharmaceutical compositions of the disclosure are formulated as shampoos and soaps to treat ringworm in humans or animals. In some embodiments, the pharmaceutical compositions of the disclosure are formulated as detergents to pre-treat or treat articles of clothing that carry fungal cells or spores.

The compositions of the disclosure can be combined with other preparations to endow the other preparations with anti-microbial properties. Non-limiting examples of preparations that can be combined with a formulation disclosed herein include Mouth Kote Dry Mouth Spray, Feminease® Feminine Moisturizer, and Pretz Nasal Spray.

### Purity of Compounds

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

### Subjects

Subjects can be, for example, elderly adults, adults, adolescents, pre-adolescents, children, toddlers, infants, and non-human animals. In some embodiments, a subject is a patient. Non-human animal subjects can be, for example, an animal (e.g., a mouse, rat, a chicken, a rabbit, a dog, a cat, a frog, or a cow). Compounds of the disclosure can be employed on surfaces in places where the spread of drug-resistant bacteria can be more likely, for example, hospitals, nursing homes, dormitories, homeless shelters, military barracks, schools, locker rooms, gymnasiums, prisons, airplanes, gyms, poultry farms, cow pens, or pigsties. The methods of the disclosure can be applied to, for example, fomites, surgical instruments, tables, chairs, doors, eating utensils, bedding, beds, and keyboards.

### Formulations

Non limiting examples of formulations of the present disclosure, i.e., MicroorGONE, are shown below in TABLE 1.

TABLE 1*

| | | | | | | Trisodium | |
|---|---|---|---|---|---|---|---|
| | CTAC | CTAB | | GLDA | | citrate | Other |
| Formulation | (mM) | (mM) | DTPA | (mM) | Solvent | (mM) | additives |
| MG | 2 | | | 20 | water | | |
| MG1 | 3 | | | 26 | water | | |
| MG2 | | 2 | | 20 | water | | |
| MG3 | | 3 | | 26 | water | | |
| MG4 | | 3 | | 20 | water | | |
| MG5 | 3 | | | 20 | water | | |
| MG6 | | | | 20 | water | 60 | 60 mM |
| | | | | | | | EDTA, 1% |
| | | | | | | | imidurea |
| MG7 | | | | 20 | water | 60 | 60 mM |
| | | | | | | | EDTA, 1% |
| | | | | | | | imidurea, 2 |
| | | | | | | | mM lauryl |
| | | | | | | | sulfate |
| MG8 | 1 | 1 | | 20 | water | 60 | 60 mM |
| | | | | | | | EDTA, 1% |
| | | | | | | | imidurea |
| MG9 | 2 | | | 8 | 0.1:99.9 | | 10 mM |
| | | | | | propylene | | EDTA, 0.2 |
| | | | | | glycol:water | | mM NAC, 1 |
| | | | | | | | mM ascorbic |
| | | | | | | | acid 10 mM |
| | | | | | | | glycine |
| MG10 | 3 | | | 10 | water | | 0.1% Tween |
| | | | | | | | 80, 1% guar |
| | | | | | | | gum, 5 mM |
| | | | | | | | glycine, |
| | | | | | | | 0.1 mM NAC |
| MG11 | 4 | | | 10 | water | | 0.1% Tween |
| | | | | | | | 80, 1% guar |
| | | | | | | | gum, 5 mM |
| | | | | | | | glycine, 2 |
| | | | | | | | mM SDS, |
| | | | | | | | 0.1 mM NAC |
| MG12 | 1 | | | 10 | water | | 1% benzoic |
| | | | | | | | acid, 1% guar |
| | | | | | | | gum, 5 mM |
| | | | | | | | glycine, 2 |
| | | | | | | | mM SDS, 0.1 |
| | | | | | | | mM NAC |
| MG13 | | 2 | | 10 | water | | 0.1% alpha |
| | | | | | | | cyclodextrin, |
| | | | | | | | 5 mM |
| | | | | | | | glycine, 2 |
| | | | | | | | mM lauryl |
| | | | | | | | sulfate, 0.1 |
| | | | | | | | mM NAC |
| MGD | 2 | | 2 | 20 | water | | |
| MGD1 | | 2 | 10 | 10 | water | | 0.1 mM |
| | | | | | | | ascorbic acid, |
| | | | | | | | 1% Tween 80 |
| MGD2 | | | 10 | 10 | water | | 10 mM SDS, |
| | | | | | | | 0.1 mM |
| | | | | | | | ascorbic acid, |
| | | | | | | | 1% Tween 80 |
| HCG | 3 | | | 26 | water | 300 | |
| G | | | | 26 | water | | |
| MG1I | 3 | | | 26 | water | | 1 mM |
| | | | | | | | imidazole |

*Abbreviations: cetyltrimethylammonium chloride (CTAC); cetyltrimethylammonium bromide (CTAB); diethylenetriaminepentaacetic acid (DTPA); N,N-dicarboxymethyl glutamic acid or a pharmaceutically-acceptable salt (GLDA); ethylenediaminetetraacetic acid (EDTA); N-acetyl-cysteine (NAC).

TABLE 2 shows comparative example formulations that do not comprise GLDA.

TABLE 2*

| | CTAC | CTAB | DTPA | | Trisodium | |
|---|---|---|---|---|---|---|
| Formulation | (mM) | (mM) | (mM) | Solvent | citrate (mM) | Other additives |
| MD | 2 | | 20 | water | | |
| C | 2 | | | water | | |
| HIMM | | | | water | 300 | |
| MDC3 | 3 | | | water | | |
| H | | | | water | 120 | |
| CC | 3 | | | water | 120 | |
| HC | 3 | | | water | 120 | |
| C1 | | | | water | | 20 mM TEA, 10 mM |
| | | | | | | glycine, 2 mM lauryl |
| | | | | | | sulfate, 1 mM NAC, |
| | | | | | | 30 mM EDTA |
| C2 | | 5 | | 1:1:98 | | 0.2 mM NAC, 10 mM |
| | | | | ethanol:propylene | | EDTA, 1 mM ascorbic |
| | | | | glycol:water | | acid |
| C3 | | | | water | | 20 mM TEA, 10 mM |
| | | | | | | glycine, 2 mM SDS, 1 |
| | | | | | | mM NAC, 30 mM |
| | | | | | | EDTA |
| C4 | | 2 | | 10:90 | | 20 mM TEA, 10 mM |
| | | | | ethanol:water | | glycine, 0.1 mM NAC, |
| | | | | | | 30 mM EDTA |
| C5 | | | 20 | water | | 1% isopropyl |
| | | | | | | myristate, 2 mM |
| | | | | | | glycine, 0.001% L- |
| | | | | | | lysine |
| C6 | | | | 10:90 | | 20 mM TEA, 10 mM |
| | | | | ethanol:water | | glycine, 2 mM SDS, |
| | | | | | | 0.1 mM NAC, 30 mM |
| | | | | | | EDTA |
| C7 | | | 20 | water | | 1% isopropyl |
| | | | | | | myristate, 2 mM |
| | | | | | | glycine, 1 mM lauryl |
| | | | | | | sulfate, 0.001% L- |
| | | | | | | lysine |
| C8 | | | | 1:99 propylene | | 5 mM SDS, 0.2 mM |
| | | | | glycol:water | | N-acetyl-cysteine, 10 |
| | | | | | | mM EDTA, 1 mM |
| | | | | | | ascorbic acid, 10 mM |
| | | | | | | glycine |
| C9 | | 2 | | water | | 20 mM TEA, 10 mM |
| | | | | | | glycine, 1 mM NAC, |
| | | | | | | 30 mM EDTA |
| C10 | | | | water | | 0.1% Tween 20, 20 |
| | | | | | | mM TEA, 10 mM |
| | | | | | | glycine, 10 mM |
| | | | | | | EDTA, 20 mM SDS, 1 |
| | | | | | | mM NAC |
| C11 | 3 | | | water | | 0.1% propylene |
| | | | | | | glycol, 5 mM SDS, 0.2 |
| | | | | | | mM NAC, 10 mM |
| | | | | | | EDTA, 1 mM ascorbic |
| | | | | | | acid, 10 mM glycine |
| C12 | | 1 | 20 | water | | 0.1% Tween 20, 20 |
| | | | | | | mM TEA, 10 mM |
| | | | | | | glycine |
| C13 | | 4 | 10 | 1:99 stearyl | | 5 mM glycine, 0.1 mM |
| | | | | alcohol:water | | NAC |
| C14 | | 2 | 10 | 1:99 stearyl | | 5 mM glycine, 2 mM |
| | | | | alcohol:water | | lauryl sulfate, 0.1 mM |
| | | | | | | NAC |

*Abbreviations: triethanolamine (TEA); N-acetyl-cysteine (NAC).

In some embodiments, a formulation of the present disclosure comprises CTAC, GLDA or a pharmaceutically-acceptable salt thereof, and water. In some embodiments, a formulation of the present disclosure comprises CTAC, GLDA or a pharmaceutically-acceptable salt thereof, DTPA or a pharmaceutically-acceptable salt, and water. In some embodiments, a formulation of the present disclosure can comprise a pH modifier (e.g., acetic acid) to adjust the pH of the formulation to about 5, about 5.4, about 6, about 6.2, about 6.5, about 6.7, about 7, about 7.4, about 7.2, about 7.5, about 7.7, 8.0 or about 8.5.

In some embodiments, the pH of the composition can have a value from about 5 to about 8.5, e.g., about 5, about 5.4, about 6, about 6.2, about 6.5, about 6.7, about 7, about 7.4, about 7.2, about 7.5, about 7.7, 8.0 or about 8.5.

In some embodiments, the composition can be diluted by 2 to 1000 times, e.g., 2×, 5×, 10×, 20×, 50×, 100×, 200×, 500×, or 1000×.

A pharmaceutical composition comprising, in unit dosage form, a formulation of the present disclosure, e.g., shown in TABLE 1 as described above, can be used to kill microorganisms. In some embodiments, a method of killing a microorganism comprises administering to the microorganism a pharmaceutical composition comprising, in unit dosage form, a formulation of the present disclosure, e.g., shown in TABLE 1 as described above. Non-limiting examples of microorganisms that can be killed via administration a pharmaceutical composition comprising, in unit dosage form, a formulation of the present disclosure, e.g., shown in TABLE 1 as described above, include bacterium, gram-positive bacterium, gram-negative bacterium, Acinetobacter baumannii, Pseudomonas aeruginosa (P. aeurginosa), Klebsiella pneumonia (K. pneumonia), Escherichia coli (E. coli), Enterobacter cloacae (E. cloacae), Burkholderia cepacia (B. cepacia), Streptococcus pyogenes, Stenotrophomonas maltophilia, Bacillus subtilis, Mycobacterium abscessus, Staphylococcus aureus (S. aureus), Staphylococcus epidermis, Propionibacterium acnes, Streptococcus mutans, Bacillus anthracis, Franscisella tularensis, Yersinia pestis, Clostridium botulinum, Coxiella burnetti, Brucella species, Burkholderia mallei, Clostridium perfringens, Vibrio cholerae, Porphyromonas gingivalis, Proteus mirabilis, Salmonella enteriditis, Shigella flexneri, Xanthomonas campestris, Clavibacter sp, fungi, Candida auris, Trichophyton rubrum, Aspergillus niger (A. niger), Candida krusei, Geomyces destructans, Trichophyton rubrum, mold, yeast, Candida albicans, Candida parapsilosis, Candida glabrata, Candida haemulonii, Candida duobshaemulonii, Candida tropicalis, Cryptococcus neoformans, Cryptococcus gattii, Batrachochytrium dendrobatidis, viruses, Lassa virus, Junin virus, Machupo virus, Ebola virus, Marburg virus, Variola major, Nipah virus, Hantavirus, Nairovirus, Varicella-zoster virus, paramyxovirus, rubeola virus, rhinovirus, coronavirus, respiratory syncytial virus, influenza virus, norovirus, hepatitis A, rotavirus, astrovirus, and Norwalk-like virus.

A pharmaceutical composition comprising, in unit dosage form, a formulation of the present disclosure, e.g., shown in TABLE 1 as described above, can be used to treat an infection in or on a subject. In some embodiments, a method of treating an infection in or on a subject comprises administering to a subject in need thereof a therapeutically-effective amount of a pharmaceutical composition comprising a formulation of the present disclosure, e.g., shown in TABLE 1 as described above. Infections treated with a pharmaceutical composition comprising, in unit dosage form, a formulation of the present disclosure, e.g., shown in TABLE 1 as described above, can be caused by, for example, microorganisms. Non-limiting examples of microorganisms that can cause an infection that can be treated via administration of a formulation of the present disclosure, e.g., shown in TABLE 1 as described above, to a subject include bacterium, gram-positive bacterium, gram-negative bacterium, Acinetobacter baumannii, Pseudomonas aeruginosa, Escherichia coli, Burkholderia cepacia, Streptococcus pyogenes, Stenotrophomonas maltophilia, Bacillus subtilis, Mycobacterium abscessus, Staphylococcus aureus, Staphylococcus epidermis, Propionibacterium acnes, Streptococcus mutans, Bacillus anthracis, Franscisella tularensis, Yersinia pestis, Clostridium botulinum, Coxiella burnetti, Brucella species, Burkholderia mallei, Clostridium perfringens, Vibrio cholerae, Porphyromonas gingivalis, Proteus mirabilis, Salmonella enteriditis, Shigella flexneri, Xanthomonas campestris, Clavibacter sp, fungi, Candida auris, Trichophyton rubrum, Aspergillus niger, Candida krusei, Geomyces destructans, Trichophyton rubrum, mold, spore, yeast, Candida albicans (C. albicans), Candida parapsilosis, Candida glabrata, Candida haemulonii, Candida duobshaemulonii, Candida tropicalis, Cryptococcus neoformans, Cryptococcus gattii, Batrachochytrium dendrobatidis, viruses, Lassa virus, Junin virus, Machupo virus, Ebola virus, Marburg virus, Variola major, Nipah virus, Hantavirus, Nairovirus, Varicella-zoster virus, paramyxovirus, rubeola virus, rhinovirus, coronavirus, respiratory syncytial virus, influenza virus, norovirus, hepatitis A, rotavirus, astrovirus, and Norwalk-like virus.

A pharmaceutical composition comprising, in unit dosage form, a formulation of the present disclosure, e.g., shown in TABLE 1 as described above, can be used to disinfect a surface. In some embodiments, a method of disinfecting a surface comprises administering to a surface in need thereof a pharmaceutical composition comprising, in unit dosage form, a formulation of the present disclosure, e.g., shown in TABLE 1 as described above. Surfaces that can be disinfected via administration of a pharmaceutical composition comprising, in unit dosage form, a formulation of the present disclosure, e.g., shown in TABLE 1 as described above, can be infected with a microorganism. Non-limiting examples of microorganisms that can infect a surface that can be disinfected via administration of a pharmaceutical composition comprising, in unit dosage form, a formulation of the present disclosure, e.g., shown in TABLE 1 as described above, include bacterium, gram-positive bacterium, gram-negative bacterium, Acinetobacter baumannii, Pseudomonas aeruginosa, Escherichia coli, Burkholderia cepacia, Streptococcus pyogenes, Stenotrophomonas maltophilia, Bacillus subtilis, Mycobacterium abscessus, Staphylococcus aureus, Staphylococcus epidermis, Propionibacterium acnes, Streptococcus mutans, Bacillus anthracis, Franscisella tularensis, Yersinia pestis, Clostridium botulinum, Coxiella burnetti, Brucella species, Burkholderia mallei, Clostridium perfringens, Vibrio cholerae, Porphyromonas gingivalis, Proteus mirabilis, Salmonella enteriditis, Shigella flexneri, Xanthomonas campestris, Clavibacter sp, fungi, Candida auris, Trichophyton rubrum, Aspergillus niger, Candida krusei, Geomyces destructans, Trichophyton rubrum, mold, yeast, Candida albicans, Candida parapsilosis, Candida glabrata, Candida haemulonii, Candida duobshaemulonii, Candida tropicalis, Cryptococcus neoformans, Cryptococcus gattii, Batrachochytrium dendrobatidis, viruses, Lassa virus, Junin virus, Machupo virus, Ebola virus, Marburg virus, Variola major, Nipah virus, Hantavirus, Nairovirus, Varicella-zoster virus, paramyxovirus, rubeola virus, rhinovirus, coronavirus, respiratory syncytial virus, influenza virus, norovirus, hepatitis A, rotavirus, astrovirus, and Norwalk-like virus.

### EXAMPLES

### Example 1: Anti-Bacterial Activity

Formulations were prepared using CTAC, DTPA, GLDA, acetic acid and water, as shown in TABLE 1 and TABLE 2. MD represents a formulation of CTAC and DTPA. MG represents a formulation of MicroorGONE. MGD represents a formulation of CTAC, GLDA and DTPA. As a control, a formulation of 2 mM CTAC was also prepared (“C”).

A lawn for each of the bacterial microorganisms was prepared on Mueler-Hinton agar (MHA) plates and spotted with 10 μL of each of the formulations MD, C, MG, and MGD. The MHA plates were further incubated at 37° C. and photographed from 24 hours to 48 hours. On the plates, F1, F2, F3, and F4 denote spot treated with MD, C, MG, and MGD, respectively.

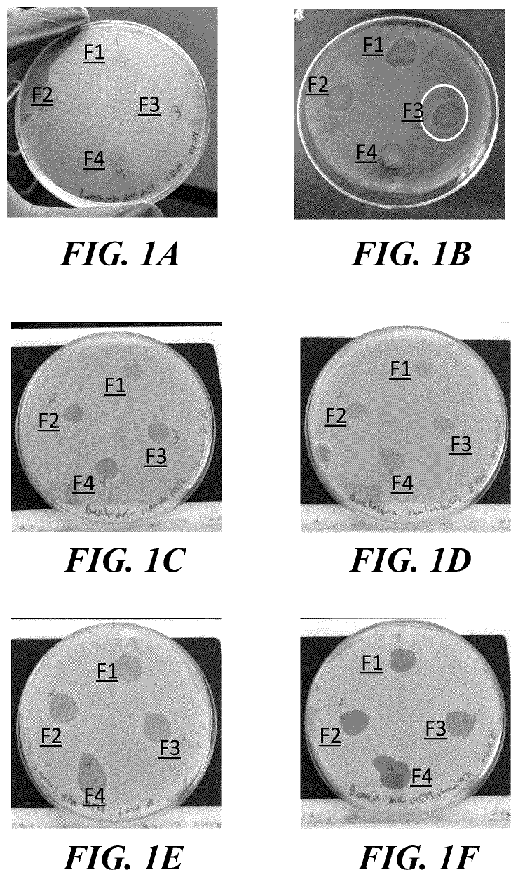

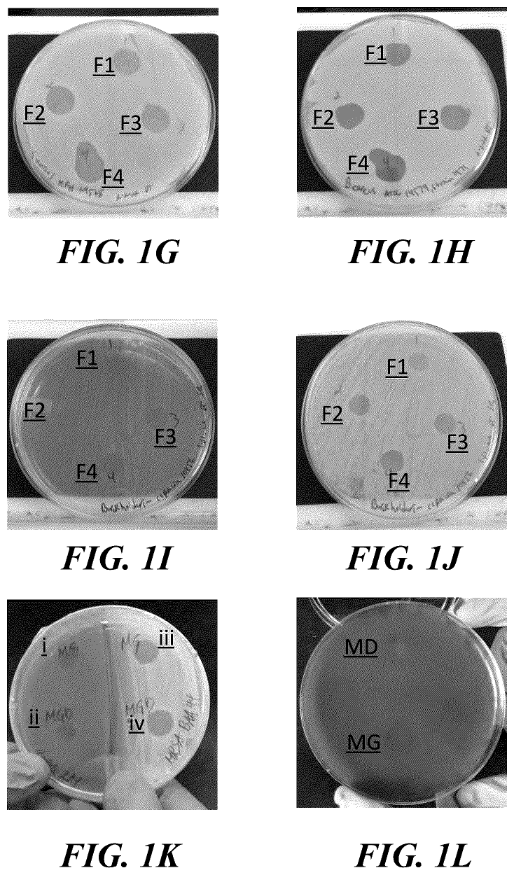

FIGS. 1A-1J show the photograph taken for each of the MHA plates. MG, C, MD, and MGD were equally effective in killing Listeria meningitides (FIG. 1B), Burkholderia cepacia ATCC 10856 (FIG. 1C), Burkholderia thailandesis E426 (FIG. 1D), S. aureus 29568 (FIG. 1E), Bacillus cereus ATCC 14579, strain 471 (FIG. 1F), Staph aureus 29568 (FIG. 1G), Bacillus cereus ATCC 14579 (FIG. 1H), Burkholderia cepacia ATCC 10856 (FIG. 1I, at 24 hr), Burkholderia cepacia ATCC 10856 (FIG. 1J, at 48 hours). However, for multidrug-resistant bacterium P. aeruginosa ATCC 2114, C was not effective, (FIG. 1A, F2) while MG was equally effective as MD and MGD.

FIG. 1K shows that MG and MGD were equally effective against multidrug-resistant P. aeruginosa ATCC 2114 and MRSA BAA 44 (spot i was P. aeruginosa treated with MG, spot ii was P. aeruginosa treated with MGD, spot iii was MRSA treated with MG, spot iv was MRSA treated with MGD). Both had full killing of the two multidrug-resistant bacteria on the plates.

In another experiment, Clostridium sporogenes, a model for the ubiquitous anaerobic C. difficile pathogen was spread over a blood agar plate and spotted with 10 μl of MD or MG. The plate was incubated at 37° C. in an anaerobic chamber for 3 days. FIG. 1L shows the photograph of the plate after 3 days and MG was equally effective against Clostridium sporogenes as MD.

These results demonstrate that MG and MGD are effective in killing broad spectrum bacteria, including multidrug-resistant bacteria.

### Example 2: Anti-fungal Activity

MG and MGD were tested in anti-fungal activity. Fugal/yeast lawn was prepared on MHA plate, spotted with 10 μl of one of the formulations and then incubated at 37° C. for 24 hours.

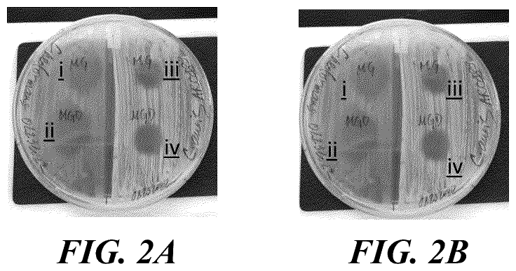

FIGS. 2A and 2B show that both MG and MGD formulations were effective against multidrug-resistant yeast C. neoformans and C. auris. The addition of DTPA can slightly enhance activity against C. neoformans (FIG. 2B). In FIG. 2A and FIG. 2B, spot i was C. neoformans treated with MG, spot ii was C. neoformans treated with MGD, spot iii was C. auris treated with MG, and spot iv was C. auris treated with MGD.

### Example 3: Sustained Protection by MicroorGONE

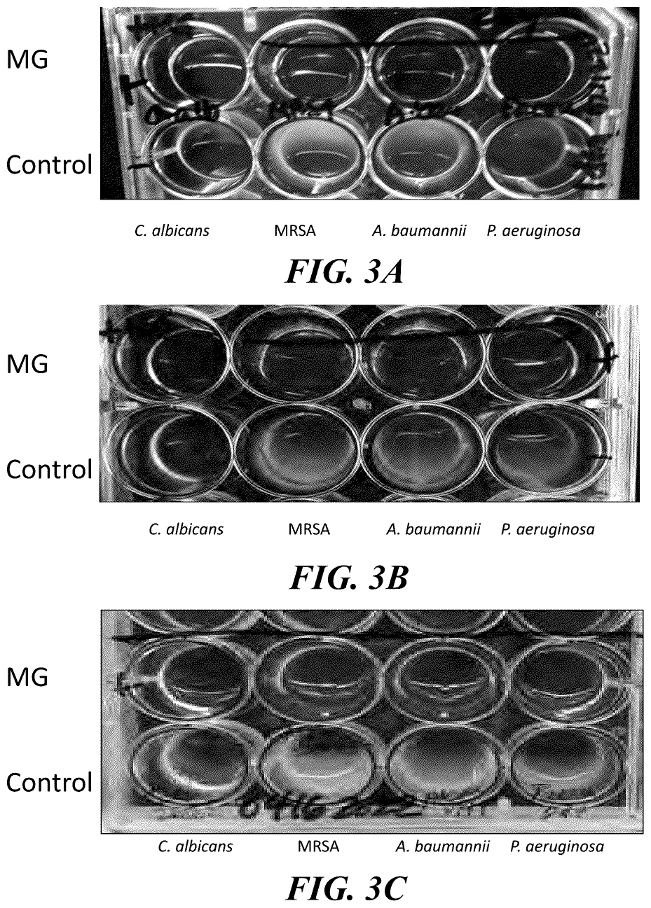

Twelve wells were filled with 250 μl MG and were left to dry. To four of the wells were added 500 μl pathogen-spoked MHB media after 24 hours to incubate C. albicans, MRSA, A. baumannii, and P. aeruginosa with one type of microorganism in one well. To four of the wells were added 500 μl pathogen-spoked MHB media after 8 days to incubate C. albicans, MRSA, A. baumannii, and P. aeruginosa with one type of microorganism in one well. To four of the wells were added 500 μl pathogen-spoked MHB media after 2 months to incubate C. albicans, MRSA, A. baumannii, and P. aeruginosa with one type of microorganism in one well. At each of the three times, four wells without MG treatment were used as controls to incubate C. albicans, MRSA, A. baumannii, and P. aeruginosa with one type of microorganism in one well. FIG. 3A shows a comparison of the wells incubated after 24 hours. FIG. 3B shows a comparison of the wells incubated after 8 days. FIG. 3C shows a comparison of the wells incubated after 2 months. Bacteria or yeast did not grow in the MG-treated well while all controls (no MG treatment) had bacteria or yeast grown in the wells.

These results demonstrate that MG formulation has sustained anti-bacterial and anti-fungal activity.

### Example 4: Toxicity to Mammalian Cells

The toxicity of MG in mammalian cells was compared to the toxicity of chlorohexidine gluconate 0.12% oral rinse using HeLa cells. HeLa cells were grown in a 5% CO₂ humidified incubator at 37° C. HeLa cells were plated in a 96-well plate to a cell density of 4000 cells/well in DMEM with 1% penicillin/streptomycin and 10% fetal bovine serum (FBS). The cells were treated with MG or chlorohexidine gluconate 0.12% oral rinse concentrations of 0.039, 0.078, 0.156, 0.313, 0.625, 1.25, 2.5, 5, and 10 μmol/L of the compounds. The cells were incubated with the compounds for 48 hr. MTT was used to assess the cell viability after the 48 hour incubation. MTT (5 mg/mL) was added to each well at a final volume of 10%, incubated for 2 hours at 37° C. in a 5% CO₂ incubator, and solubilized in 100 μL of DMSO before the absorbance was read at 595 nm.

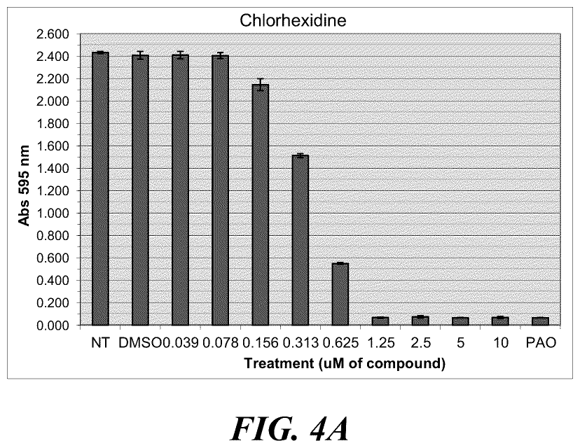

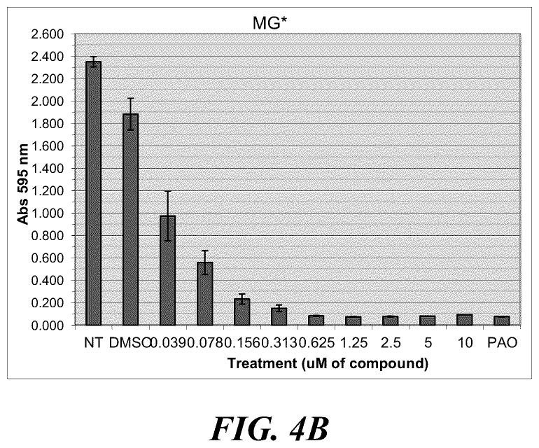

FIG. 4A shows the toxicity of chlorohexidine gluconate 0.12% oral rinse against HeLa cells. Chlorohexidine gluconate 0.12% oral rinse had an IC₅₀ (GI₅₀) of 0.402% v/v of the compound. FIG. 4B shows the toxicity of MG against HeLa cells. MG had an IC₅₀ (GI₅₀) of 0.039% v/v of the compound. TABLE 3 shows the IC₅₀ (% v/v) and SD % of the formulations.

TABLE 3

| Formulation | GI₅₀ (% v/v) | SD % |
|---|---|---|
| MG | 0.039 | 4.8 |
| chlorohexidine gluconate | 0.22 | 0.4 |
| 0.12% oral rinse | | |

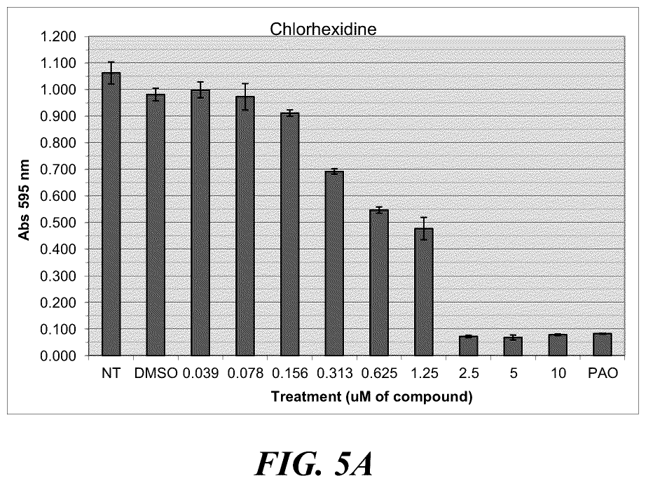

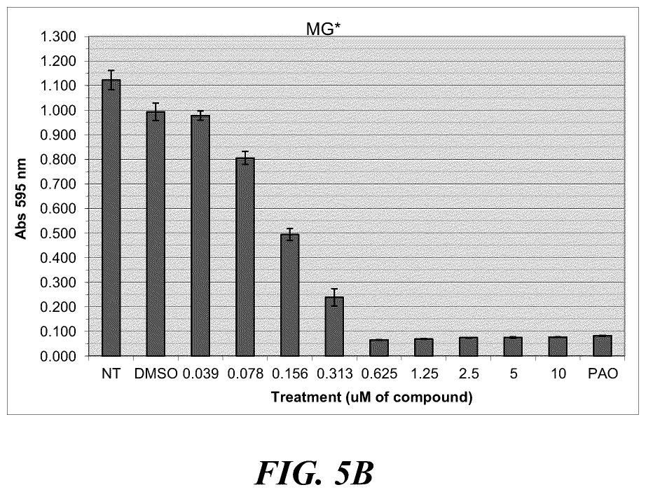

The same tests were done with ARPE-19 (human retinal pigment cells). FIG. 5A shows the toxicity of chlorohexidine gluconate 0.12% oral rinse against ARPE-19 cells. Chlorohexidine gluconate 0.12% oral rinse had an IC₅₀ (GI₅₀) of 0.658% v/v of the compound. FIG. 5B shows the toxicity of MG against ARPE-19 cells. MG had an IC₅₀ (GI₅₀) of 0.145% v/v of the compound. TABLE 4 shows the IC₅₀ (% v/v) and SD % of the formulations.

TABLE 4

| Formulation | GI₅₀ (% v/v) | SD % |
|---|---|---|
| MG | 0.145 | 0.46 |
| chlorohexidine gluconate | 0.658 | 1.67 |
| 0.12% oral rinse | | |

Those tests demonstrate that MG is less toxic than chlorohexidine gluconate 0.12% oral rinse is against several types of mammalian cells.

### Example 5: Activity Against Fungi

A sparse fungal lawn was prepared on MHA plate and spotted with 10 μL of the following formulations: MG (2 mM CTAC+20 mM GLDA), Chx (Chlorhexidine Oral 0.12% Solution) and F (Fungizone®, 250 μg/mL of Amphotericin B).

TABLE 5 shows that MG killed all 15 strains of the fungal pathogens. Chx exhibited less killing of the fungal pathogens C. parapsilosis AR 0335, C. lusitaniae AR 0398, and C. glabrata AR 0326. F exhibited less killing of the fungal pathogens C. duobushaemuloni AR 0391 and C. haemuloni AR 0393. “++” indicates antifungal activity with complete clearing and “+” indicates antifungal activity with some clearing.

TABLE 5

| Microorganism | MG | Chx | F |
|---|---|---|---|
| C. parapsilosis AR 0344 | ++ | ++ | ++ |
| C. parapsilosis AR 0339 | ++ | ++ | ++ |
| C. parapsilosis AR 0335 | ++ | + | ++ |
| C. lusitaniae AR 0398 | ++ | + | ++ |
| C. glabrata AR 0326 | ++ | + | ++ |
| C. krusei AR 0397 | ++ | ++ | ++ |
| C. glabrata AR 0318 | ++ | ++ | ++ |
| C. albicans ATCC 26555 | ++ | ++ | ++ |
| C. glabrata AR 0314 | ++ | ++ | ++ |
| C. auris AR 0390 | ++ | ++ | ++ |
| C. duobushaemuloni AR 0391 | ++ | ++ | + |
| Kodameae ohmeri AR 0396 | ++ | ++ | ++ |
| C. auris AR 0383 | ++ | ++ | ++ |
| C. auris AR 0381 | ++ | ++ | ++ |
| C. haemuloni AR 0393 | ++ | ++ | + |

### Example 6: Activity Against Fungi

Fungal lawn was spread over MHA plate and spotted with 10 um of the following formulations: MG1 (3 mM CTAC+26 mM GLDA) and MG1I (3 mM CTAC+26 mM GLDA+1 mM imidazole).

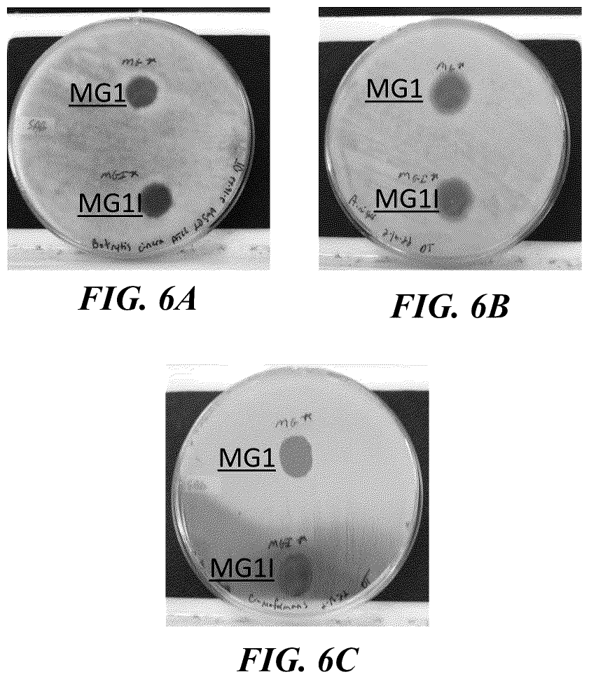

FIG. 6A shows the killing activity of both formulations against Botrytis cinera ATCC 20599. FIG. 6B shows the killing activity of both formulations against Aspergillus niger. FIG. 6C shows the killing activity of both formulations against Cryptococcus neoformans. These tests demonstrate that both formulations were effective against these two fungal pathogens. The presence of imidazole neither hindered nor increased activity of the formulation.

### Example 7: Anti-Bacterial Activity and Invasion Prevention

A MRSA lawn was prepared on MHA plate and spotted with 10 μl of the following formulations: MG (2 mM CTAC+20 mM GLDA) and Mup (Mupirocina®).

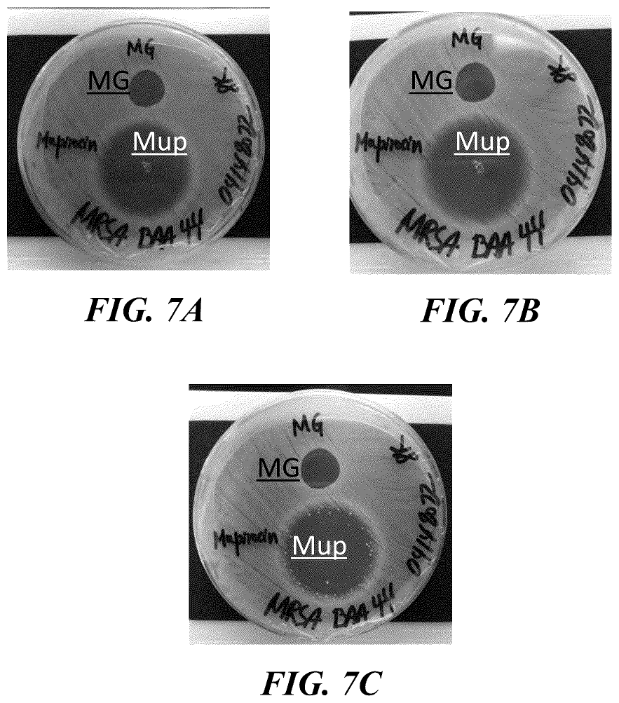

FIG. 7A shows the plate at 24 hours after treatment with both formulations. FIG. 7B shows the plate at 2 days after treatment with both formulations. FIG. 7C shows the plate at 8 days after treatment with both formulations. Mupirocin was initially more active (covered larger area) but over the next few days, the fuzzy border grew in with resistant colonies. In contrast, the size of MG clearance did not change and remained resistant to invasion.

### Example 8: Anti-Bacterial Activity

Bacterial lawn was prepared on MHA plate and spotted with 10 μl of the following formulations: MG1, Mup (Mupirocin), Baci (Equate™ Bacitracin Zinc), Neo (Neomycin®), Triple (Equate™ Triple Antibiotic Ointment).

TABLE 6 shows that MG killed all 13 strains of the microorganisms, while Mup, Baci, Neo and Triple had less killing or no killing in at least 10 of the microorganisms. “++” indicates complete clearing; “+” indicates some clearing; and “−” indicates nearly no clearing.

TABLE 6

| Microorganism | MG1 | Mup | Baci | Neo | Triple |
|---|---|---|---|---|---|
| Strep A | ++ | ++ | + | + | − |
| P. aeruginosa ATCC 27853 | ++ | + | + | + | + |
| S. pyogenes | ++ | ++ | − | ++ | ++ |
| Vancomycin resistant | ++ | − | − | − | − |
| enterococcus | | | | | |
| VRE ATCC 51299 | | | | | |
| Salmonella infantis AR-0920 | ++ | + | − | + | − |
| Yesenia pestis Kim 6 | ++ | ++ | − | + | + |
| Burkholderia cepacia ATCC | ++ | + | − | + | − |
| 10856 | | | | | |
| Klebsiella pneumoniae CRE | ++ | − | − | + | + |
| ATCC 1705 | | | | | |
| E. coli O157 | ++ | + | − | + | + |
| P. aeruginosa ATCC 15692 | + | − | − | + | + |
| P. mirabilis | ++ | + | − | + | + |
| Salmonella infantis AR-0919 | ++ | + | − | + | + |
| M. smegmatis ATCC 607 | ++ | − | − | ++ | ++ |

### Example 9: Evaluating the Efficacy of MicroorGONE as a Disinfectant Using a Time-Kill Kinetic Assay

The disinfectant properties of MicroorGONE were evaluated on each of the bacterial and yeast species. All organisms were grown in MH broth for 12 hours in a rotary shaking incubator (100 rpm) at 37° C. 1.6 mL Eppendorf tubes were prepared as follows: 1) 90 μL of Letheen neutralizing buffer (LET blocking buffer; 5 g/L beef extract, 0.7 g/L lecithin, 5 g/L polysorbate 80, and 5 g/L sodium chloride); 2) 90 μL MicroorGONE; and 3) 90 μL LET and 10 μL of undiluted cell suspension (ranging from 1×10⁵ to 1×10⁸ CFU/mL) were added to each of the above described tubes and incubated either 30 seconds, 2 minutes, or 10 minutes at room temperature. After the incubation, 10 μL of cell suspension was removed from 2) and re-suspended into 3) to neutralize the toxicity of MicroorGONE. The samples were placed into a CytoOne 96-well microtiter plate (USA Scientific, Orlando, FL), diluted 10-fold, and drip-streaked onto 100 mm×15 mm Mueller-Hinton agar (MHA) plates (Simport Scientific Inc., Saint-Mathieu-de-Beloeil, QC). The MHA plates were incubated for 12 hours at 37° C. and the efficacy of the disinfectant properties of MicroorGONE were evaluated in terms of colony forming units with respect to the population of remaining viable bacteria and yeast.

TABLE 7 shows the results of the experiment (all ran in triplicates). The data show that MG eradicated a starting population of cells of about 10⁵ to about 10⁸ pathogens within less than 2 minutes of exposure for most of the microorganisms except P. mirabilis and B. cepacia ATCC 10856 which MG eradicated within less than 10 minutes of exposure.

TABLE 7

| | Challenge | | | Post-exposure | | |
|---|---|---|---|---|---|---|
| | suspension | Exposure | | Population | Log₁₀ | Percent |
| Microorganism | (CFU/mL) | Time | | (CFU/mL) | Reduction | Reduction |
| MDR A. baumannii | 3.37 × 10⁷ ± 5.44 × 10⁶ | 30 | seconds | 0 | 7 | 100% |
| ATCC BAA-1797 | | | | | | |
| MRSA ATCC BAA-44 | 1.87 × 10⁸ ± 9.43 × 10⁶ | 30 | seconds | 0 | 8 | 100% |
| MDR P. aeruginosa | 4.90 × 10⁸ ± 1.31 × 10⁸ | 30 | seconds | 0 | 8 | 100% |
| ATCC BAA-2114 | | | | | | |
| Candida albicans | 2.07 × 10⁵ ± 4.19 × 10⁴ | 30 | seconds | 0 | 5 | 100% |
| ATCC 26555 | | | | | | |
| E. coli O157:H7 | 1.67 × 10⁷ ± 8.33 × 10⁶ | 2 | minutes | 0 | 7 | 100% |
| Carbapenem-resistant | 2.50 × 10⁷ ± 8.83 × 10⁶ | 2 | minutes | 0 | 7 | 100% |
| Enterobacteriaceae | | | | | | |
| (CRE) | | | | | | |
| ATCC BAA-1705 | | | | | | |
| Streptococcus pyogenes | 3.03 × 10⁷ ± 7.84 × 10⁶ | 30 | seconds | 0 | 7 | 100% |
| 85W1180 | | | | | | |
| Salmonella infantis | 2.03 × 10⁷ ± 5.91 × 10⁶ | 2 | minutes | 0 | 7 | 100% |
| AR0919 | | | | | | |
| Vancomycin- resistant | 8.10 × 10⁵ ± 4.19 × 10⁵ | 30 | seconds | 0 | 5 | 100% |
| Enterococci (VRE) | | | | | | |
| ATCC 51299 | | | | | | |
| P. mirabilis | 2.47 × 10⁷ ± 3.09 × 10⁶ | 10 | minutes | 0 | 7 | 100% |
| B. cepacia ATCC 10856 | 1.70 × 10⁷ ± 5.71 × 10⁶ | 10 | minutes | 0 | 7 | 100% |
| Y. pestis Kim 6 | 5.57 × 10⁶ ± 2.29 × 10⁶ | 30 | seconds | 0 | 6 | 100% |

### Example 10: Anti-Bacterial Activity of MicroorGONE Against Colistin Resistant Bacteria

MicroorGONE formulations (MG, MG1, and HCG) were tested against colistin (polymyxin E, or PME) resistant bacterium S. maltophilia, in comparison to formulations MD, H, C, MDC3, CC, and HC. The formulations are shown in TABLE 1 and TABLE 2.

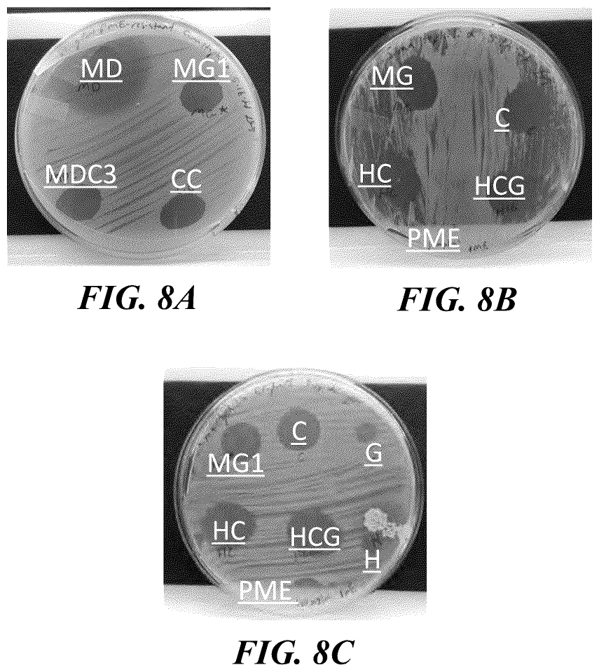

The bacterial lawns were prepared on MHA plates. The bacterial lawn was spotted by 10 μL of the formulation and the plate was then incubated at 37° C. for 2 days. FIGS. 8A-8C show photographs of the plates after 2 days incubation. CTAC formulations (C in FIG. 8B and FIG. 8C, CC in FIG. 8A) were active against S. maltophilia. Trisodium citrate or GLDA alone did not work as well as CTAC. However, MicroorGONE formulations (MG in FIG. 8B and MG1 in FIG. 8C) also had full kill of S. maltophilia. FIGS. 8B and 8C also show that Trisodium citrate did not interfere with CTAC or MicroorGONE formulations.

### Example 11: Antiviral Properties of MicroorGONE

An overnight grow of E. coli XL1 was inoculated with a small amount of MS2 phage ATCC 15597-B1 from a frozen glycerol stock and incubated at 37° C. for 24 hours. The mixture was agitated by vortex to mix and then spun by centrifuge for 5 minutes at 5,000 rpm to pellet the E. coli. The supernatant was filtered with a 0.45 μm filter to collect the MS2 phages. Upon collection, 10-fold serial dilutions of MS2 phages were made in sterile molecular water.

For testing, 50 μL of the diluted MS2 phage (10⁻⁵ dilution) was treated with 50 μL sterile water (mixture 1), 50 μL MicroorGONE MG1 (mixture 2, 50% v/v final concentration), 50 μL MicroorGONE HIMM (mixture 3, 50% v/v final concentration), or 50 μL MicroorGONE HCG (mixture 4, 50% v/v final concentration). The mixtures were then incubated for 10 minutes at room temperature. Following the 10-minute incubation, each of the 100 μL mixture 1, mixture 2, mixture 3, and mixture 4 was added to a 1 mL of the overnight grow of E. coli XL1 and 4 mL 0.9% soft TSA.

The resulting solutions were mixed, poured on MHA, and incubated for 18 hours at 37° C. Plaque counts were performed following the incubation. Triplicates were performed for each formulation.

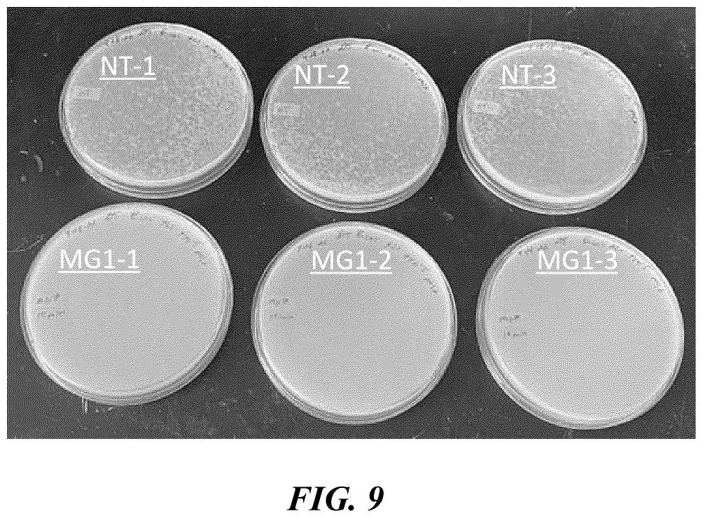

FIG. 9 shows that the 3 plates with no MG1 treatment (NT-1, NT-2, and NT-3) had nearly no MS2 phages kill. The 3 plates with MG1 treatments (MG1-1, MG1-2, and MG1-3) had full kill of MS2 phages with a 10 min treatment of MG1.

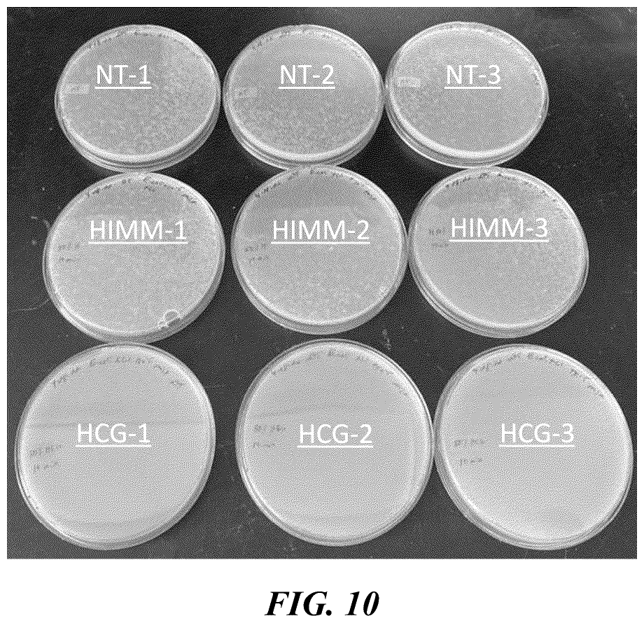

FIG. 10 shows that the 3 plates with no MG1 treatment (NT-1, NT-2, and NT-3) and the 3 plates with HIMM treatments had nearly no MS2 phages kill. The 3 plates with HCG1 treatments (HCG1-1, HCG1-2, and HCG1-3) had full kill of MS2 phages with a 10 min treatment of HCG1. These results demonstrate the antiviral activity of MicroorGONE formulations.

### Example 12: Anti-Microbial Activity of MicroorGONE with Tween 80 (Polysorbate)

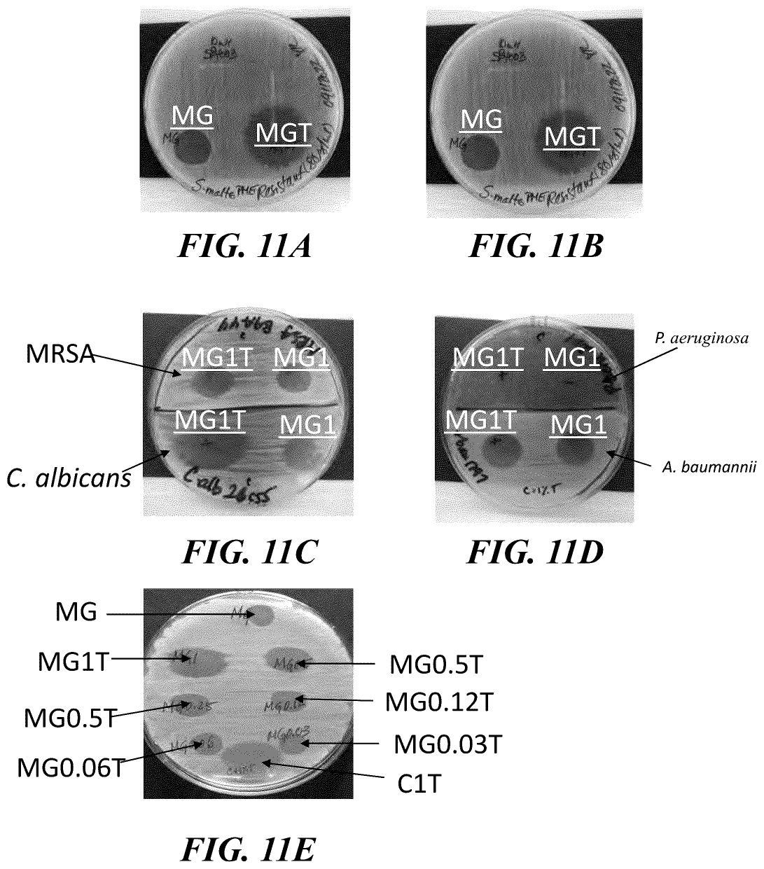

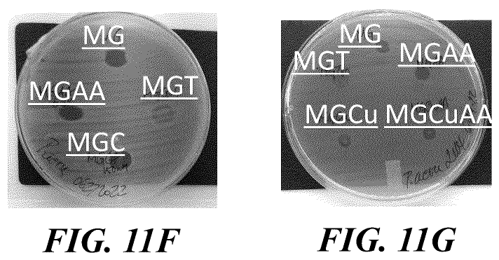

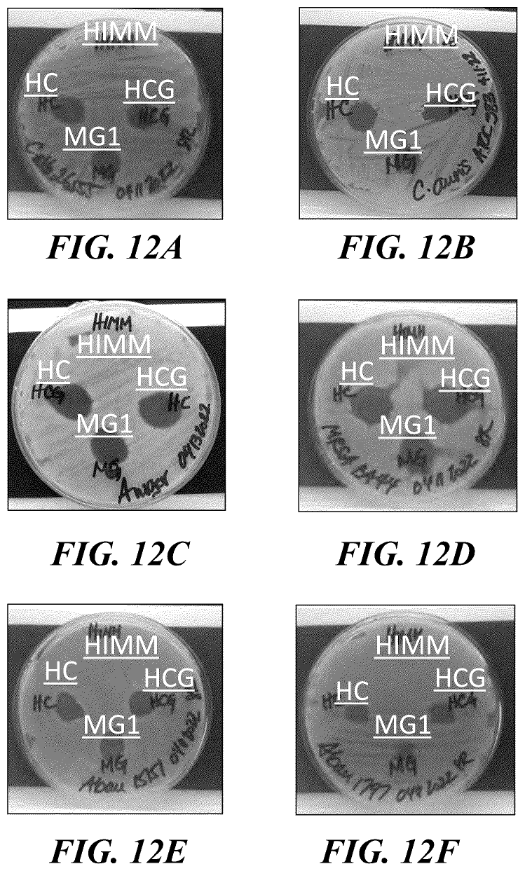

Anti-microbial activity of MicroorGONE was evaluated in combination with Tween 80 (polysorbate). A lawn was prepared with Bacillus subtilis on MHA plate, spotted with 10 μl of MG or MGT (MG with 1% v/v Tween 80), and then incubated at 37° C. for overnight. FIG. 11A shows that MG activity against Bacillus subtilis largely increased in the presence of Tween 80.

A lawn of S. maltophilia ATCC 13637 strain, previously evolved to be resistant to >80 μg/ml of PME, was prepared on MHA plate, spotted with 10 μl of MG or MGT, and then incubated at 37° C. for overnight. FIG. 11B shows that MG activity against S. maltophilia ATCC 13637 largely increased in the presence of Tween 80.

Lawns were prepared with MRSA, C. albicans, P. aeruginosa, and A. baumannii on MHA plates, spotted with 10 μl of MG1 or MG1T (MG1 with 1% v/v Tween 80), then incubated at 37° C. for overnight. FIG. 11C shows that MG1 activity against C. albicans increased in the presence of Tween 80 while MG1 activity against MRSA remained the same. FIG. 11D shows that MG1 activity against P. aeruginosa, and A. baumannii remained the same in the presence of Tween 80.

The effect of concentration of Tween 80 was also tested. Lawns prepared with C. albicans on MHA plates, spotted with 10 μl of MG, MG1T (MG with 1% v/v Tween 80), MG0.5T (MG with 0.5% v/v Tween 80), MG0.25T (MG with 0.25% v/v Tween 80), MG0.12T (MG with 0.12% v/v Tween 80), MG0.06T (MG with 0.06% v/v Tween 80), MG0.03T (MG with 0.03% v/v Tween 80), or CIT (2 mM CTAC and 1% v/v Tween 80), then incubated at 37° C. for overnight. FIG. 11E shows that as the concentration of Tween 80 in the formulation increased from 0.03% to 1%, the enhancement of MG activity against C. albicans also increased. FIG. 11E also shows that Tween 80 improved activity against C. albicans of CTAC formulation.

For MG formulation, the addition of Tween 80 slightly reduced the activity of MG against P. aeruginosa. FIG. 11F shows that the MGT (MG with 1% v/v Tween 80) had slightly less clearing of P. aeruginosa as compared with MG. MG formulation with ascorbic acid (MGAA, with 0.2% ascorbic acid), citrate (MGC, with 150 mM trisodium citrate), copper (MGCu, with 1 mM Cu²⁺), and copper ascorbic acid (MGCuAA, comprising 1 mM CuSO₄ and 0.2% ascorbic acid) were also tested in this experiment. FIGS. 11F and 11G show that neither ascorbic acid nor citrate had any inhibition of MG activity against P. aeruginosa. FIG. 11G shows that copper inhibited MG activity against P. aeruginosa.

### Example 13: Trisodium Citrate Based MicroorGONE

Trisodium citrate is a mild chelating agent that can work with CTAC/CTAB. Trisodium citrate (HIMM) based MicroorGONE formulations (HC=300 mM trisodium citrate+3 mM CTAC and HCG=300 mM trisodium citrate+3 mM CTAC+26 mM GLDA) were assessed for antibacterial and anti-fungal activity. The formulations are shown in TABLE 1 and TABLE 2.

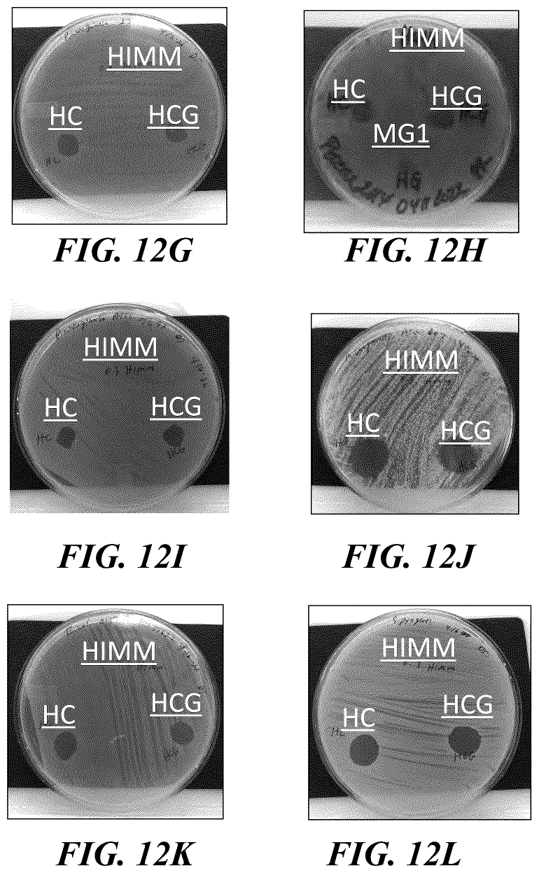

FIGS. 12A-12C show HIMM alone was not effective in killing fungal pathogens Candida albicans ATCC 26555 (FIG. 12A), Candida auris ATCC 383 (FIG. 12B), Aspergillus niger (FIG. 12C). However, when used with CTAC (HC), and MG1 (HCG), the formulations had full kill of the three fungal pathogens. Trisodium citrate did not interfere with the surfactant CTAC or chelating agent GLDA.

FIGS. 12D-12L show HIMM alone was not effective in killing bacterial pathogens MRSA BAA-44 (FIG. 12D), A baumannii 15151 (FIG. 12E), MDR A. baumannii 1797 (FIG. 12F), P. aeruginosa ATCC 27601 (FIG. 12G), MDR P. aeruginosa ATCC BAA-2114 (FIG. 12 H), P. aeruginosa ATCC 15692 (FIG. 12I), Mycobacterium smegmatis ATCC 607 (FIG. 12J), E. coli O157 (FIG. 12K), and S. pyogenes Strep A (FIG. 12L), but when used with CTAC (HC) and MG1 (HCG), the formulations exhibited full kill of the bacterial pathogens.

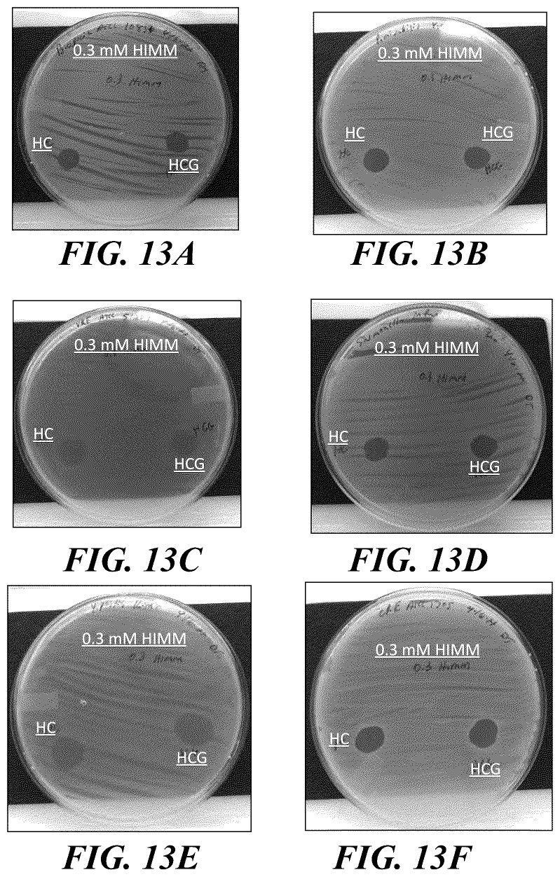

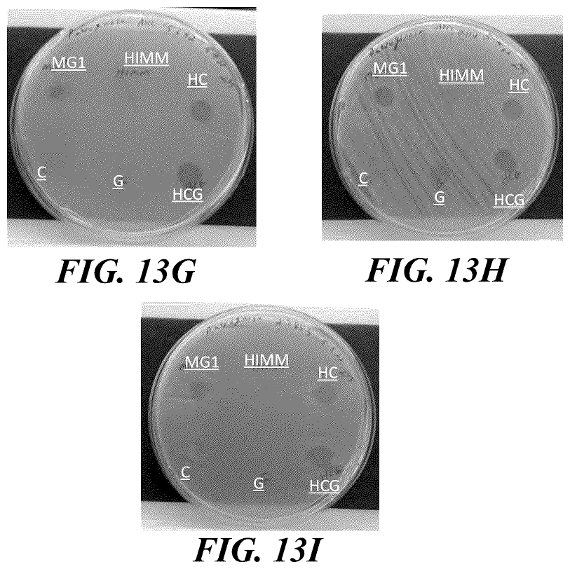

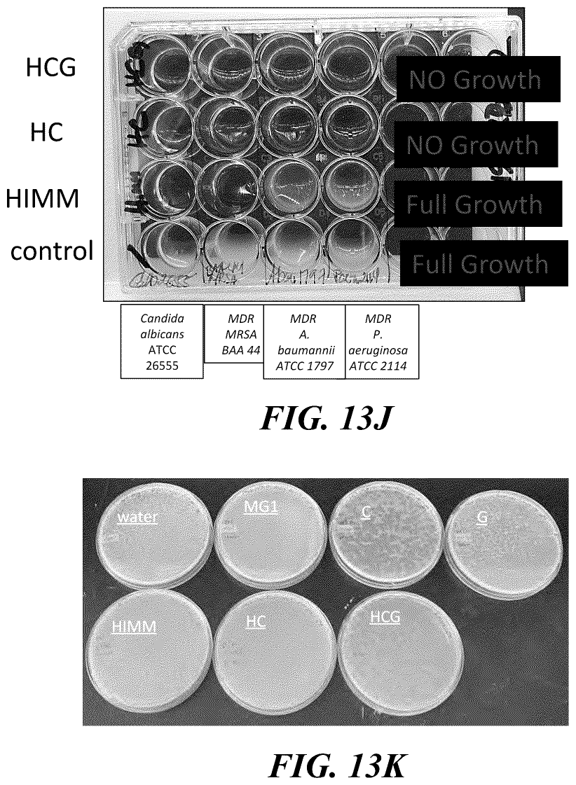

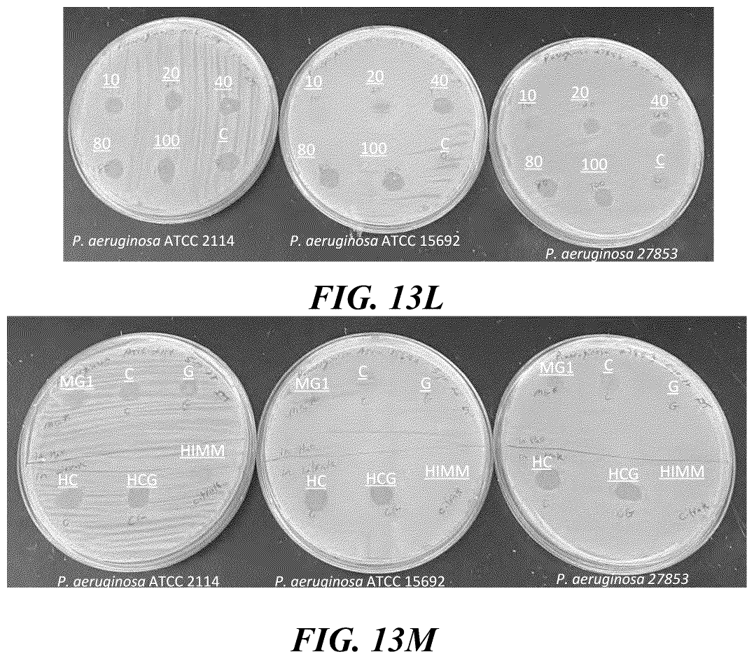

FIGS. 13A-13I show HIMM alone was not effective in killing bacterial pathogens Burkholderia cepacia ATCC 10856 (FIG. 13A), Proteus mirabilis (FIG. 13B), Vancomycin-resistant enterococcus VRE ATCC 51299 (FIG. 13C), MDR Salmonella infantis AR0920 (FIG. 13D), Yesenia pestis Kim 6 (FIG. 13E), Klebsiella pneumoniae CRE ATCC 1705 (FIG. 13F), P. aeruginosa ATCC 15692 (FIG. 13G), P. aeruginosa ATCC 2114 (FIG. 13H), P. aeruginosa 27853 (FIG. 13I), but when used with CTAC (HC) and MG1 (HCG), the formulations exhibited full kill of the bacterial pathogens.

FIG. 13J shows growth pattern of Candida albicans ATCC 2655, MDR MRSA BAA 44, MDR A. baumannii ATCC 1797, and MDR P. aeruginosa ATCC 2114 in presence of HIMM, HC, and HCG, and in a control from left to right. The pathogens grew in the absence of any additive (control) and in the presence of HIMM-coated surface but are inhibited from growing when HIMM contained CTAC or MG.

The effect of HIMM concentration in the HC (HIMM+3 mM CTAC) was evaluated. In FIG. 13L, 10, 20, 40, 80, and 100 represent HC formulation with HIMM of 30 mM, 60 mM, 120 mM, 240 mM, and 300 mM. FIG. 13L shows a HC formulation with HIMM reduced to 120 mM is still effective against P. aeruginosa strains ATCC 2114, ATCC 15692, and 27853.

FIG. 13M shows HC and HCG are the most effective formulations against P. aeruginosa strains ATCC 2114, ATCC 15692, and 27853.

The anti-viral activity of formulations MG1, C, G, HIMM, HC, and HCG was also evaluated. Water was used as a control. An overnight grow of E. coli XL1 was inoculated with a small amount of MS2 phage ATCC 15597-B1, then incubated at 37° C. for 24 hours. The solution was vortexed and centrifuged for 5 minutes at 5,000 rpm. MS2 phages were collected by filtering the supernatant with 0.45 μm filter. The MS2 phages were diluted by 5 times 10-fold serial dilutions. 50 μL of the phage dilution was added to an eppi tube. Then 50 μL water, 50 μL MG1, 50 μL C, 50 μL G, 50 μL HIMM, 50 μL HC, or 50 μL HCG was introduced to the eppi tube and the mixture was incubated for 10 minutes. Subsequent to the treatment, the 100 μL solutions and 1 mL of an overnight grow of E. coli XL1 were added to 4 mL of 0.9% soft TSA, poured on MHA and incubated 18 hours at 37° C. PFUs were counted following the incubation. FIG. 13K shows that MG1 and HC had the largest population of E. coli XL1, indicating the killing of MS2 phases by the MG1 and HC formulations. Formulations C, G, HIMM, and HCG were not as effective as MG1 and HC.

### Example 14: MicroorGONE with Ascorbic Acid

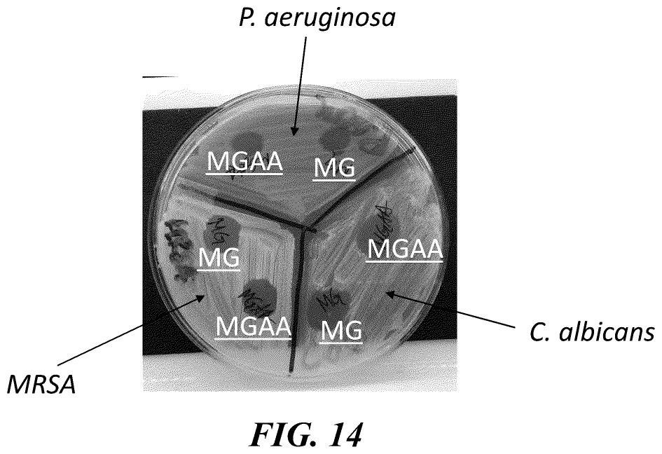

Ascorbic acid was added to MG formulation to test if ascorbic acid inhibits the activity of MG. FIG. 14 shows that ascorbic acid with a concentration of 0.2% w/v did not inhibit MG activity against C. albicans, P. aeruginosa and MRSA.

### Example 15: Activity of MicroorGONE Against Spores

Spores were prepared and harvested with a modified method from Cairns et al. (Quantitative phenotypic screens of Aspergillus niger mutants in solid and liquid culture. STAR Protoc. 2022 Dec. 16; 3(4): 101883, which is incorporated herein by reference in its entirety).

Sabouraud Dextrose Agar (SDA) plate was inoculated with Aspergillus niger (A. niger) and incubated at 30° C. for 5 days. Spores were collected from the plate by performing a sterile cotton swab over the spores and were immersed into sterile water. Spores and the sterile water were mixed thoroughly by vortex. The mixture was then spun by centrifuge for 1 minute at 15,000 rpm. After removing the supernatant, the spores were re-suspended into sterile water to form a stock solution.

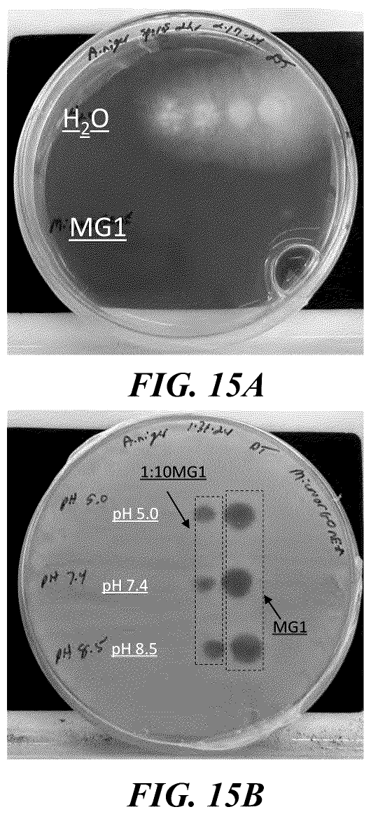

10 μL stock solution was added to 100 μL of sterile water or MicroorGONE formulation (MG1 as shown in Table 1) in a tube respectively, and the resulting mixtures were incubated at room temperature for 2 hours. After the incubation period, the tubes containing the treated spores were agitated by vortex for 30 seconds at 15,000 rpm. The supernatant was removed and the tube was washed 3× with 100 μL sterile Letheen broth. The solid phase (e.g., precipitated spores) was re-suspended in 100 μL of sterile water and diluted 10-fold with sterile water. 10 μL of the diluted solution was spotted onto SDA plate and the plate was incubated at 30° C. for 2 days. FIG. 15A shows the plate spotted with spores treated with MicroorGONE (“MG1”) and water (“H₂O”). No A. niger growth was observed at the locations on the plates that were spotted with spores treated with MG1, indicating MG1 killed the spores and prevented the growth of spores. Large A. niger growth was observed at the locations on the plates that were spotted with spores treated with water only (e.g., no MG1 treatment). MicroorGONE exhibited killing activity against A. niger spores, with about 4-orders of magnitude reduction of spore compared with water.

Activity of MG1 with different pH values against A. niger was evaluated. A lawn of A. niger spores collected from a stock plate was spread onto an SDA plate with sterile cotton swab. MG1 solutions with pH value of 5.0, 7.4, and 8.5 were prepared and diluted 10-fold in sterile water. 5 μL of the MG1 and diluted MG1 (1:10 MG1) solutions were spotted on the SDA plates and the plates were incubated at 30° C. for 3 days. FIG. 15B shows an image of the plate treated with MG1 and 1:10 MG1 at different pH value. The spotted areas were clear with no visible A. niger growth. Full strength MicroorGONE and diluted MicroorGONE maintain effectiveness at all tested pH values. The MicroorGONE formulations possess profound activity against fungal spores.

### Example 16: Antibacterial and Antifungal Activities of MicroorGONE at Different pH

10 mL MHB was inoculated with bacteria (P. aeruginosa ATCC 27853, S. aureus ATCC 29213, B. cepacia ATCC 10856) or yeast C. albicans ATCC 26555) cultures and incubated at 37° C. for 18 hours. A lawn of bacteria or yeast (200 μL from the overnight grow) was spread on MHA plate. MicroorGONE (MG1 as shown in Table 1) was prepared at pH of 5, 7.4, or 8.5 and diluted 10-fold in sterile water. 5 μL of each MG1 solution was spotted on respective MHA plates that were inoculated with bacteria or yeast. The plates were then incubated at 37° C. for 18 hours.

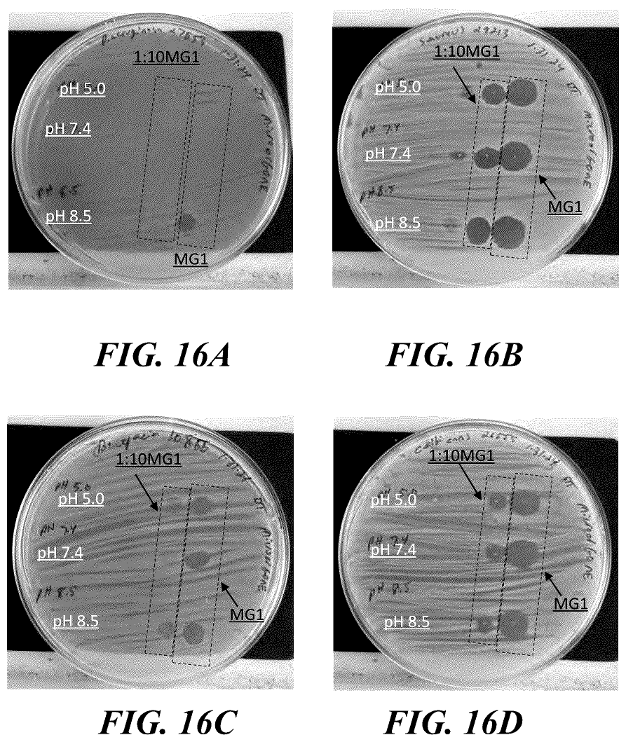

FIGS. 16A-16D show images of the MHA plates inoculated with P. aeruginosa (FIG. 16A), S. aureus (FIG. 16B), B. cepacia (FIG. 16C), and C. albicans (FIG. 16D), that were spotted with MG1 and diluted MG1 (1:10 MG1) solutions at different pH values. Full strength MicroorGONE (MG1, no dilution) maintained effectiveness against S. aureus, B. cepacia, and C. albicans at all three pHs tested. Full strength MicroorGONE was effective against the thick lawn of P. aeruginosa at pH 8.5. 1:10 MG1 remains effective against S. aureus and C. albicans at all three pHs. The MicroorGONE formulations possess profound activity against multi-drug resistant bacteria and fungi across a very wide spectrum of pH.

### Example 17: Antimicrobial Activities of MicroorGONE with P&G

The antimicrobial activities of MicroorGONE (MG1 as shown in Table 1) with P&G (Procter & Gamble General Cleaner product) against B. cepacia ATCC 10856, E. coli, P. aeurginosa ATCC 27853, K. pneumonia ATCC BAA-2342, S. aureus ATCC 29213), C. albicans ATCC 26555, E. cloacae ATCC BAA-2341, Aspergillus niger, and pooled inoculum (B. cepacian, E. coli, P. aeruginosa, K. pneumonia, S. aureus, and C. albicans) were evaluated following the procedures shown below:

Procedure 1: the microorganism was isolation streaked onto Mueller-Hinton agar (MHA) plate from frozen stock. The plates were incubated at a temperature for a period of time as shown in Table 8.

Procedure 2: the microorganism was collected by rolling a sterile cotton-tipped swab over confluent lawns and transferred into a 5 mL sterile tube containing sterile 0.85% NaCl saline.

Procedure 3: the tube was agitated by vortex and the inoculum was adjusted to percent transmittance at 425 nm (% T_{425 nm}) of between 23-25 for S. aureus, between 31-33 for B. cepacia, F. coli, P. aeurginosa, and K. pneumonia, and between 0.2-0.4 for C. albicans. 100 μL of the % T_{425 nm} adjusted S. aureus, B. cepacian, E. coli, P. aeruginosa, K. pneumonia, and C. albicans cultures were mixed into a 1.6 mL sterile tube to form a pooled inoculum.

Procedure 4: the % T_{425 nm} adjusted individual inoculum and pooled inoculums were quantified by preparing 10⁰, 10⁻¹, 10⁻², 10⁻³, 10⁻⁴, 10⁻⁵, 10⁻⁶, and 10⁻⁷ dilutions using Mueller-Hinton broth (MHB) through serial 10-fold dilutions in a 96-well plate. 10 μL of the dilutions were drip streaked onto a MHA plate, and the plate was incubated at a temperature for a period of time as shown in Table 8.

Procedure 5: testing samples were prepared by adding 250 μL of sterile 0.85% NaCl saline (control), 250 μL of P&G (PG), 250 μL P&G with MicroorGONE (PG+MG1), 250 μL P&G with 1:10 diluted MG1 (PG+1:10 MG1), or 250 μL P&G with 1:100 diluted MG1 (PG+1:100 MG1) into 1.6 mL sterile tubes. 1:100 dilution of the previously adjusted inoculums (individuals and pooled) was added to the tubes and the samples were incubated at a temperature for a period of time as shown in Table 8. After the treatments, the microbial concentration was quantified using a similar procedure as described in Procedure 4.

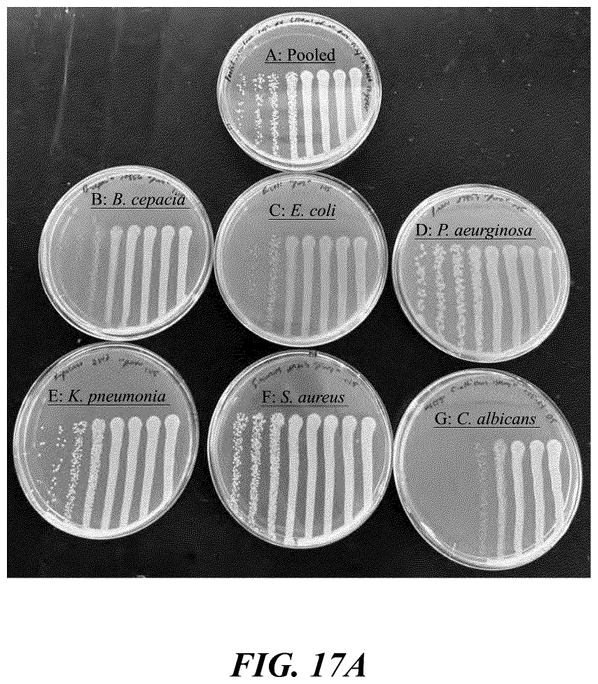

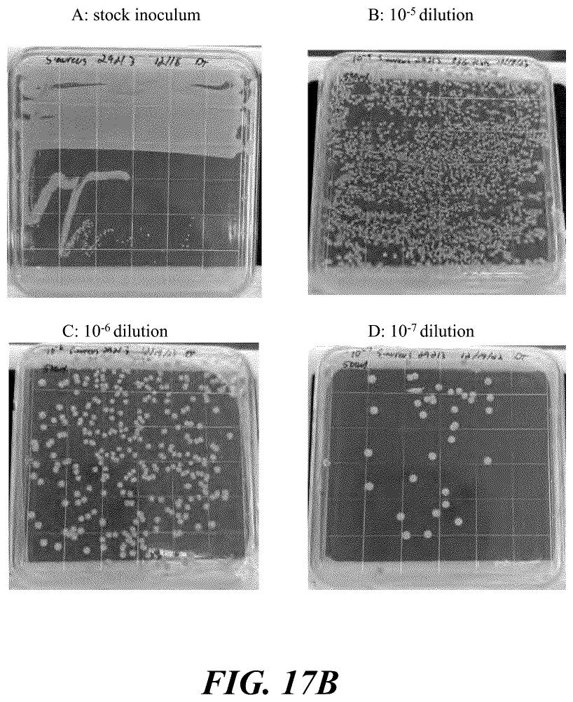

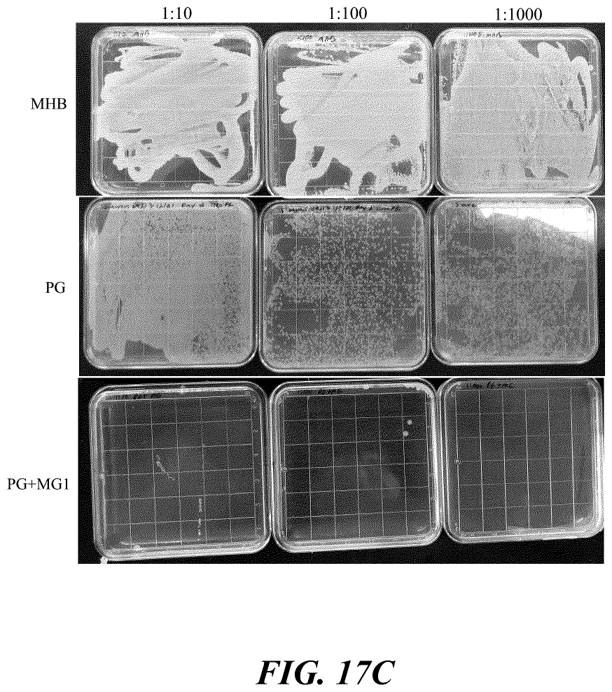

FIG. 17A shows the quantification of different inoculums (PANEL A: pooled, PANEL B: B. cepacian, PANEL C: E. coli, PANEL D: P. aeruginosa, PANEL E: K. pneumonia, PANEL F: S. aureus, and PANEL G: C. albicans) using MHB before the treatments.

FIG. 17B shows the quantification of S. aureus stock inoculum (PANEL A) and after dilutions (PANELS B-D). S. aureus ATCC 29213 was isolation streaked onto Mueller-Hinton agar (MHA) plate from frozen stock and the plate was incubated at 37° C. for 18 hours. Cells were collected by rolling a sterile cotton-tipped swab over confluent bacterial lawn. The collected cells were transferred into a 5 mL sterile tube containing sterile 0.85% NaCl saline. The tube was agitated by vortex and the inoculum was adjusted to % T_{425 nm} of 25. The adjusted test inoculum was quantified by preparing 10⁻⁵, 10⁻⁶, and 10⁻⁷ dilutions using Mueller-Hinton broth (MHB). 500 μL of the dilutions were poured and spread onto MHA plates and the plates were incubated at 37° C. for 18 hours. Stock inoculum was quantified as being 8×10⁹ CFU/mL, with test inoculum being 8×10⁷CFU/mL. Testing samples were prepared by adding 250 μL of sterile MHB, 250 L of PG, or 250 μL PG+MG1 in 1.6 mL sterile tubes. 1:100 dilution of the adjusted stock inoculum was added to each of the tubes and the samples were incubated at 25° C. for 2 days. Following the 2 day incubation, 1:10, 1:100, and 1:1000 dilutions using Mueller-Hinton broth (MHB) were made for each of the samples (MHB, PG, and PG+MG1). 500 μL of the dilutions were poured and spread onto MHA plates and the plates were incubated at 30° C. for 3 days before the quantification. FIG. 17C shows the plates with S. aureus treated with MHB, PG, and PG+MG1 at different dilutions (e.g., 1:10, 1:100, and 1:1000). MHB served as positive growth control and plates were covered with large colonies of S. aureus, even at 1:1000 dilution. PG alone resulted in less growth than MHB control, but the plate still contained about 2.1×10⁶ CFU/mL of S. aureus, which is about 1-log reduction from the starting concentration. Treatment with PG+MG1 resulted in full bacterial kill (i.e., 7-log reduction).

The activity against A. niger was performed following the procedure shown below.

Procedure 1: A. niger was isolation streaked onto SDA plate from frozen stock. The plate was incubated at 30° C. for 8 days.

Procedure 2:10 mL of sterile Saline with Tween80 was poured onto the surface of the SDA plate. The resultant A. niger on the SDA surface was gently agitated using a sterile swab and the suspension was collected into a sterile container and mixed thoroughly.

Procedure 3: The cells were then transferred into a 5 mL sterile tube containing sterile 0.85% NaCl saline.

Procedure 4: the inoculum was quantified by preparing 10⁰, 10⁻¹, 10⁻², 10⁻³, 10⁻⁴, and 10⁻⁵ dilutions using sterile 0.85% NaCl saline through serial 10-fold dilutions in a 96-well plate. 10 μL of the dilutions were drip streaked onto a SDA plate and the plate was incubated at 30° C. for 2 days. Stock inoculum was quantified as 7×10⁷ CFU/mL, with a test inoculum of 7×10⁵ CFU/mL (1:100 dilution).

Procedure 5: testing samples were prepared by adding 250 μL of sterile 0.85% NaCl saline (control), 250 μL of P&G (PG), 250 μL P&G with MicroorGONE (PG+MG1), 250 μL P&G with 1:10 diluted MG1 (PG+1:10 MG1), or 250 μL P&G with 1:100 diluted MG1 (PG+1:100 MG1) into 1.6 mL sterile tubes. 1:100 dilution (7×10⁵ CFU/mL) of the stock inoculum was added to the tubes and the samples were incubated at 30° C. for 2 days. After the treatments, the microbial concentration was quantified using a similar procedure as described in Procedure 4.

TABLE 8
Testing parameters

| | | | | | Testing sample | |
|---|---|---|---|---|---|---|
| | | | | | microbial | |
| | Incubation on | | Incubation on | Microbial | concentration | Incubation on |
| | MHA plate | | MHA plate | concentration | (1:100 dilution) | MHA plate |
| Organism | (Procedure 1) | % T_{425 nm} | (Procedure 4) | (CFU/mL) | CFU/mL | (Procedure 5) |
| S. aureus | 37° C. for 18 hr | 32 | 37° C. for 18 hr | 2.6 × 10⁸ | 2.6 × 10⁶ | 37° C. for 2 days |
| ATCC 29213 | | | | | | |
| P. aeurginosa | 37° C. for 18 hr | 31 | 37° C. for 18 hr | 1.08 × 10¹¹ | 1.08 × 10⁹ | 37° C. for 2 days |
| ATCC 27853 | | | | | | |
| 1^{I} | | | | | | |
| P. aeurginosa | 37° C. for 24 hr | 34 | 37° C. for 24 hr | 2.4 × 10⁸ | 2.4 × 10⁶ | 37° C. for 2 days |
| ATCC 27853 | | | | | | |
| 2^{II} | | | | | | |
| E. coli | 37° C. for 18 hr | 32 | 37° C. for 18 hr | 3.5 × 10⁸ | 3.5 × 10⁶ | 37° C. for 2 days |
| C. albicans | 30° C. for 2 days | 0.8 | 30° C. for 2 days | 3.2 × 10⁷ | 3.2 × 10⁵ | 25° C. for 2 days |
| ATCC 26555 | | | | | | |
| 1^{III} | | | | | | |
| C. albicans | 30° C. for 2 days | 0.4 | 30° C. for 2 days | 2.6 × 10⁸ | 2.6 × 10⁶ | 37° C. for 2 days |
| ATCC 26555 | | | | | | |
| 2^{IV} | | | | | | |
| B. cepacia | 37° C. for 24 hr | 33 | 37° C. for 24 hr | 5.3 × 10⁸ | 5.3 × 10⁶ | 37° C. for 2 days |
| ATCC 10856 | | | | | | |
| K. pneumonia | 37° C. for 18 hours | 31 | 37° C. for 18 hours | 4.8 × 10⁸ | 4.8 × 10⁶ | 37° C. for 2 days |
| ATCC BAA- | | | | | | |
| 2342 | | | | | | |
| E. cloacae | 37° C. for 18 hr | 31 | 37° C. for 18 hr | 5.1 × 10⁸ | 5.1 × 10⁶ | 37° C. for 2 days |
| ATCC BAA- | | | | | | |
| 2341 | | | | | | |
| Aspergillus | 30° C. for 8 days | | 30° C. for 2 days | 7 × 10⁷ | 7 × 10⁵ | 30° C. for 2 days |
| niger | | | | | | |
| Pooled | 37° C. for 24 hr | | 37° C. for 24 hr | 2.5 × 10¹⁰ | 2.5 × 10⁸ | 37° C. for 2 days |
| inoculum 1^{V} | | | | | | |
| Pooled | 37° C. for 24 hr | | 37° C. for 24 hr | 2.5 × 10¹⁰ | 2.5 × 10⁸ | 37° C. for 8 days |
| inoculum 2^{VI} | | | | | | |

^{I}250 μL P&G with MicroorGONE (MG1) was tested
^{II}250 μL P&G with MicroorGONE (MG1) was not tested
^{III}C. albicans inoculated at 25° C. for 2 days in procedure 5
^{IV}C. albicans inoculated at 37° C. for 2 days in procedure 5
^{V}pooled inoculum inoculated for 2 days in procedure 5
^{VI}pooled inoculum inoculated for 8 days in procedure 5

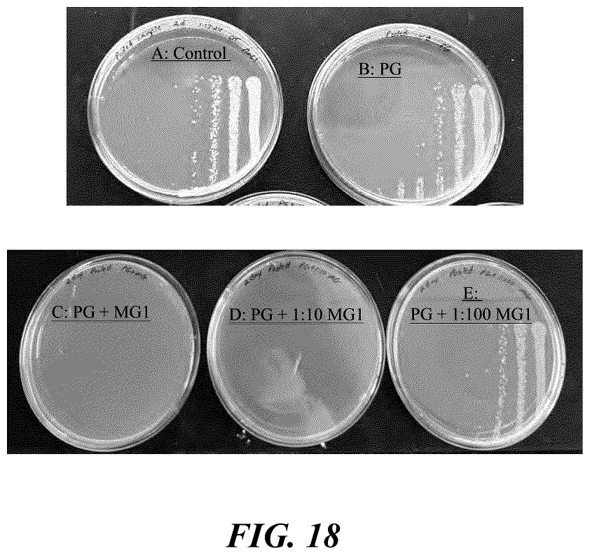

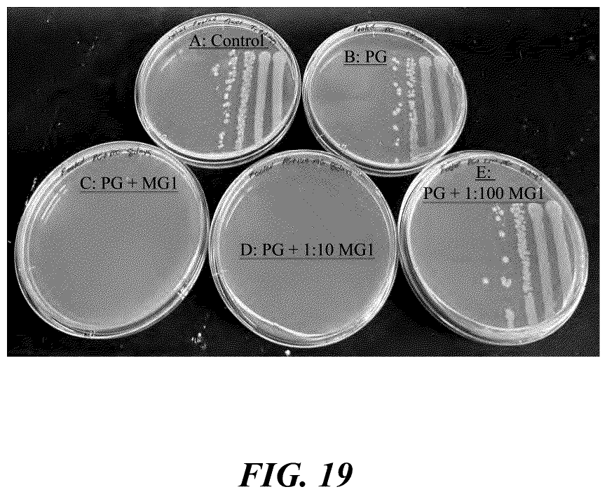

FIG. 18 shows the quantification of the pooled inoculums treated with different compositions for 2 days (PANEL A: Control, PANEL B: PG, PANEL C: PG+MG1, PANEL D: PG+1:10 MG1, PANEL E: PG+1:100 MG1). FIG. 19 shows the quantification of the pooled inoculums treated with different compositions for 8 days (PANEL A: Control, PANEL B: PG, PANEL C: PG+MG1, PANEL D: PG+1:10 MG1, PANEL E: PG+1:100 MG1). The microbial quantification results are shown in Table 9. PG treatment led to a 1-log (1 order of magnitude) reduction of pooled inoculum in comparison to the Control. Treatment with PG+MG1 or PG+1:10 MG1 resulted in full kill of the microorganisms in the pooled inoculum. Treatment with PG+1:100 MG1 had similar growth reduction as compared with PG alone.

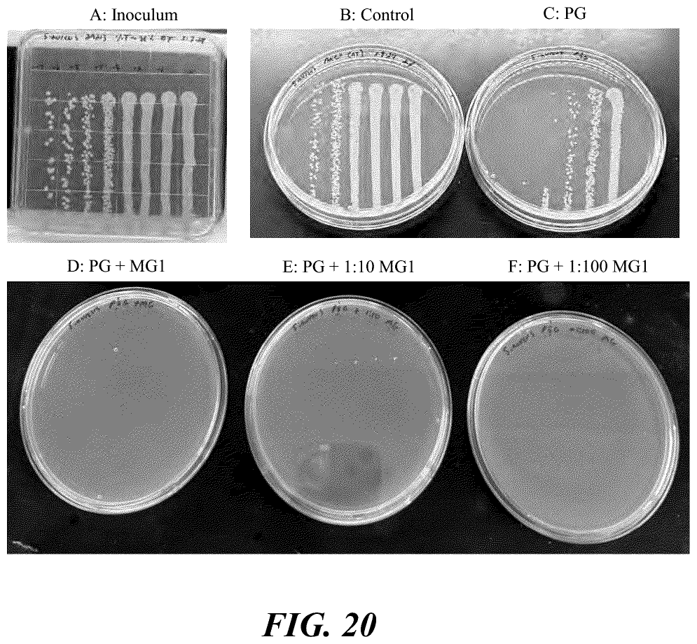

FIG. 20 PANEL A shows the quantification of S. aureus stock inoculum. The stock inoculum has a concentration of 2.6×10⁸ CFU/mL. FIG. 20 PANELS B-F show the quantification of S. aureus treated with different compositions (PANEL B: Control, PANEL C: PG, PANEL D: PG+MG1, PANEL E: PG+1:10 MG1, PANEL F: PG+1:100 MG1). The microbial quantification results are shown in Table 9. PG treatment led to a 2-log reduction. Treatment with PG+MG1, PG+1:10 MG1, or PG+1:100 MG1 resulted in full kill of S. aureus.

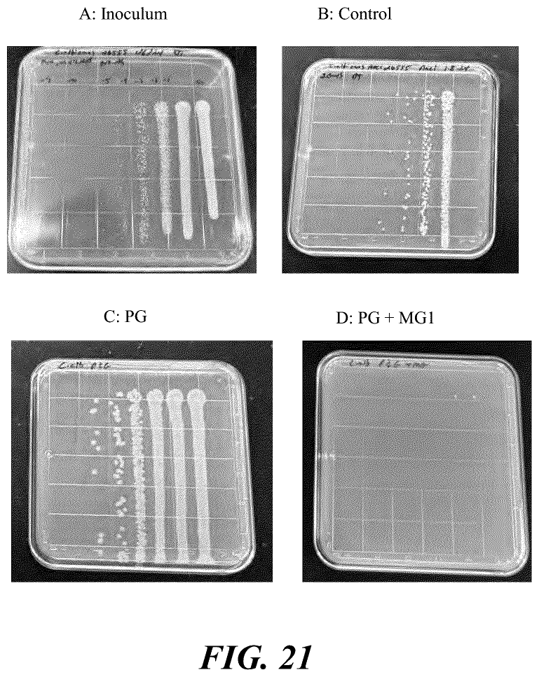

FIG. 21 PANEL A shows the quantification of C. albicans 1 stock inoculum. The stock inoculum has a concentration of 3.2×10⁷ CFU/mL. FIG. 21 PANELS B-D show the quantification of C. albicans 1 treated with different compositions (PANEL B: Control, PANEL C: PG, PANEL D: PG+MG1). The microbial quantification results are shown in Table 9. Bacterial contaminant was present in PG treated sample that inoculated at 25° C. Treatment with PG+MG1 resulted in full kill of C. albicans and the potential bacterial contaminant.

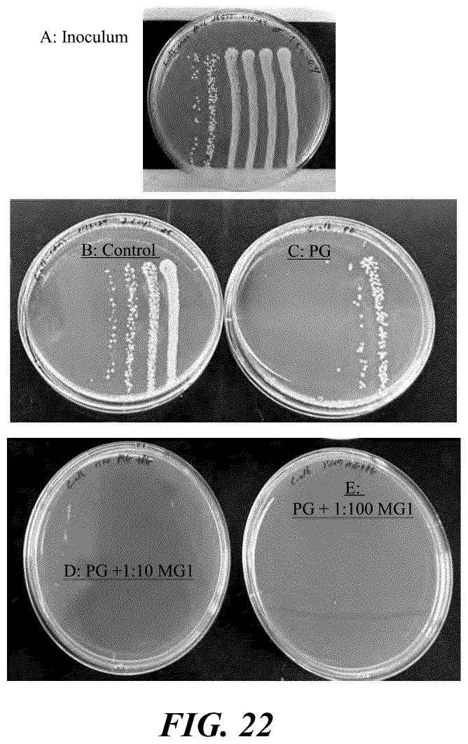

FIG. 22 PANEL A shows the quantification of C. albicans 2 stock inoculum. The stock inoculum has a concentration of 2.6×10⁸ CFU/mL. FIG. 22 PANELS B-E show the quantification of C. albicans 2 treated with different compositions (PANEL B: Control, PANEL C: PG, PANEL D: PG+1:10 MG1, PANEL E: PG+1:100 MG1). The microbial quantification results are shown in Table 9. PG treatment led to a 2-log reduction. No bacterial contamination was observed. Treatment with PG+1:10 MG1 or PG+1:100 MG1 resulted in full kill of C. albicans. The bacterial contaminant occurred at incubation at 25° C. but not at incubation at 37° C., indicating the bacterium may be a temperature-sensitive strain. PG solutions were plated on MHA plates and samples were swabbed from the plates and incubated at 25° C. The colonies were bright green, implying the contaminant could be a strain of pseudomonas. The bacteria were fluorescent under UV light. The bacteria may be of P. aeruginosa origin. Gram-stain determination indicated that the bacteria were gram-negative rods. Susceptibility testing against the PG bacterial contaminant revealed that the bacteria were resistant to penicillin, bacitracin, vancomycin, and ampicillin but susceptible to kanamycin and streptomycin. The contaminant(s) may be mixed strains. Further evaluation can be done to identify the origin and strain of the contaminant(s).

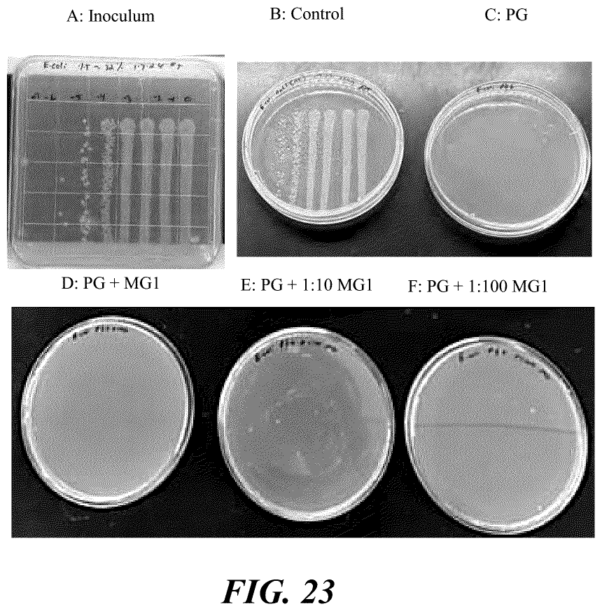

FIG. 23 PANEL A shows the quantification of E. coli stock inoculum. The stock inoculum has a concentration of 3.5×10⁸ CFU/mL. FIG. 23 PANELS B-F show the quantification of E. coli treated with different compositions (PANEL B: Control, PANEL C: PG, PANEL D: PG+MG1, PANEL E: PG+1:10 MG1, PANEL F: PG+1:100 MG1). The microbial quantification results are shown in Table 9. Treatment with PG, PG+MG1, PG+1:10 MG1, or PG+1:100 MG1 resulted in full kill of E. coli.

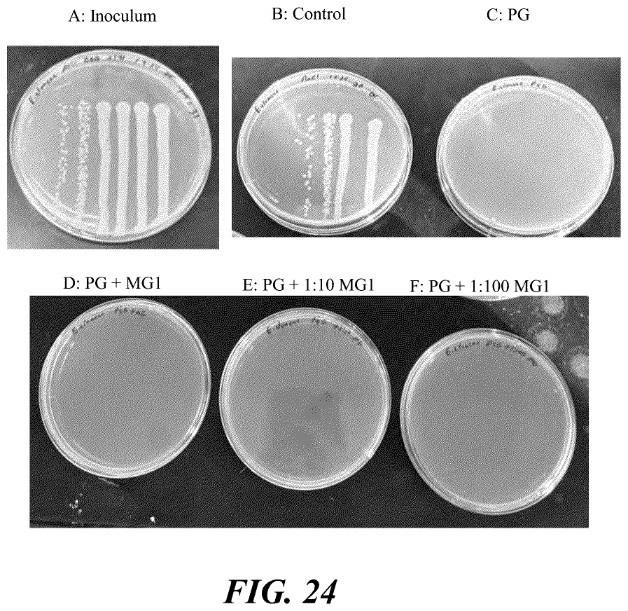

FIG. 24 PANEL A shows the quantification of E. cloacae stock inoculum. The stock inoculum has a concentration of 5.1×10⁸ CFU/mL. FIG. 24 PANELS B-F show the quantification of E. cloacae treated with different compositions (PANEL B: Control, PANEL C: PG, PANEL D: PG+MG1, PANEL E: PG+1:10 MG1, PANEL F: PG+1:100 MG1). The microbial quantification results are shown in Table 9. Treatment with PG, PG+MG1, PG+1:10 MG1, or PG+1:100 MG1 resulted in full kill of E. cloacae.

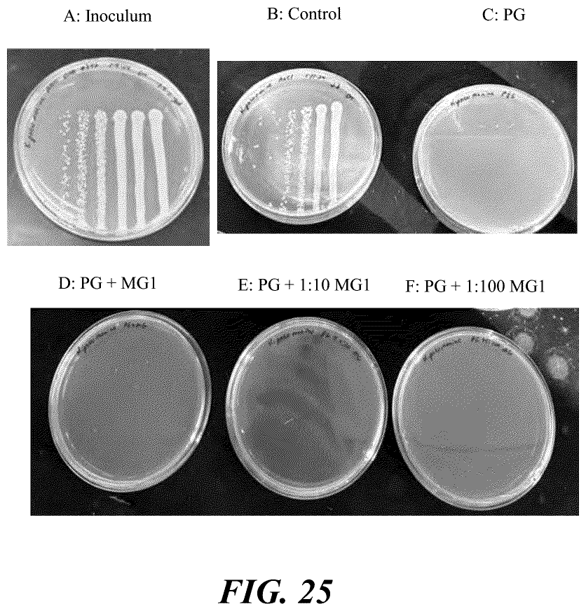

FIG. 25 PANEL A shows the quantification of Klebsiella pneumoniae stock inoculum. The stock inoculum has a concentration of 4.8×10⁸ CFU/mL. FIG. 25 PANELS B-F show the quantification of Klebsiella pneumoniae treated with different compositions (PANEL B: Control, PANEL C: PG, PANEL D: PG+MG1, PANEL E: PG+1:10 MG1, PANEL F: PG+1:100 MG1). The microbial quantification results are shown in Table 9. Treatment with PG, PG+MG1, PG+1:10 MG1, or PG+1:100 MG1 resulted in full kill of Klebsiella pneumoniae.

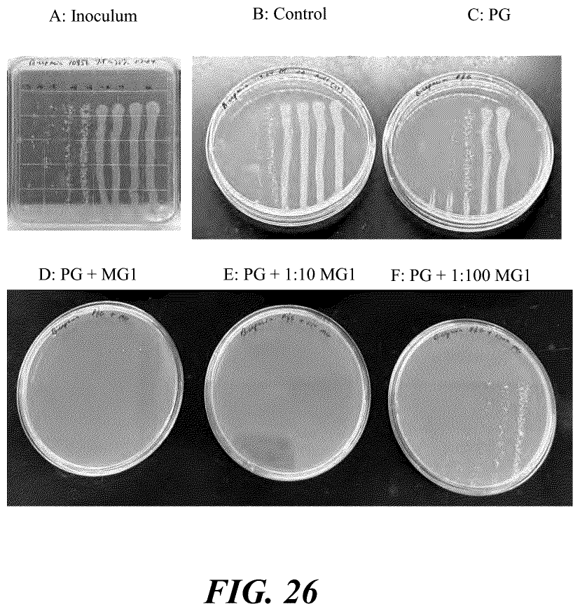

FIG. 26 PANEL A shows the quantification of Burkholderia cepacia stock inoculum. The stock inoculum has a concentration of 5.3×10⁸ CFU/mL. FIG. 26 PANELS B-F show the quantification of Burkholderia cepacia treated with different compositions (PANEL B: Control, PANEL C: PG, PANEL D: PG+MG1, PANEL E: PG+1:10 MG1, PANEL F: PG+1:100 MG1).

The microbial quantification results are shown in Table 9. PG did not kill Burkholderia cepacia but inhibited the growth. Treatment with PG+MG1 or PG+1:10 MG1 resulted in full kill of Burkholderia cepacia. Treatment with PG+1:100 MG1 resulted in 1-log reduction compared with treatment with PG.

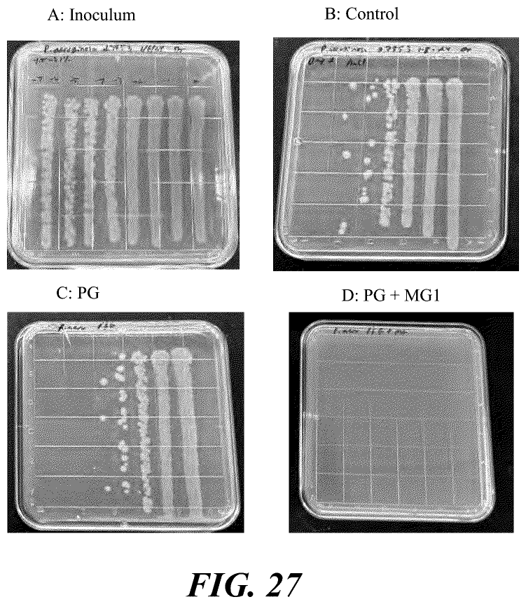

FIG. 27 PANEL A shows the quantification of P. aeurginosa 1 stock inoculum. The stock inoculum has a concentration of 1.08×10¹¹ CFU/mL. FIG. 27 PANELS B-D show the quantification of P. aeurginosa 1 treated with different compositions (PANEL B: Control, PANEL C: PG, PANEL D: PG+MG1). The microbial quantification results are shown in Table 9. Treatment with PG led to 1-log reduction of P. aeurginosa 1. Treatment with PG+MG1 resulted in full kill of P. aeurginosa 1.

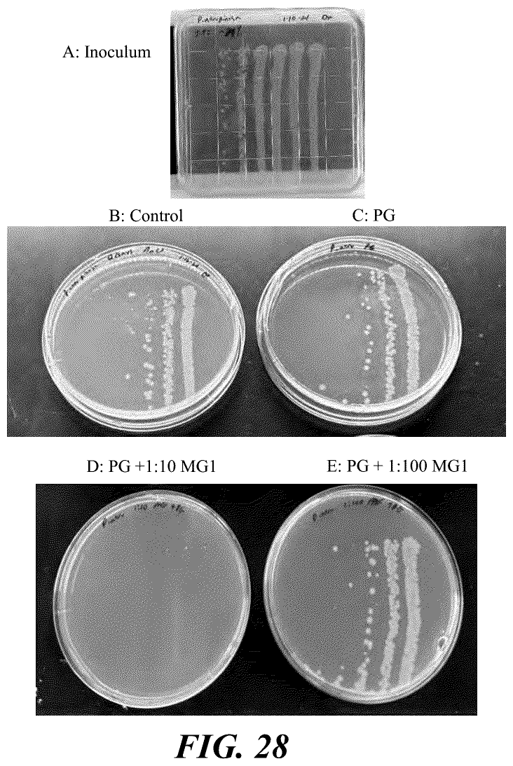

FIG. 28 PANEL A shows the quantification of P. aeurginosa 2 stock inoculum. The stock inoculum has a concentration of 2.4×10⁸ CFU/mL. FIG. 28 PANELS B-E show the quantification of P. aeurginosa 2 treated with different compositions (PANEL B: Control, PANEL C: PG, PANEL D: PG+1:10 MG1, PANEL E: PG+1:100 MG1). The microbial quantification results are shown in Table 9. Treatment with PG had minimal reduction of P. deurginosa 2. Treatment with PG+1:10 MG1 resulted in full kill of P. aeurginosa 2.

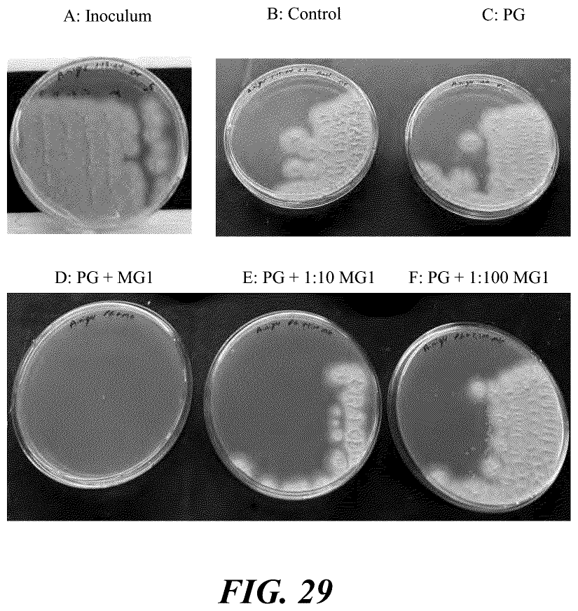

FIG. 29 PANEL A shows the quantification of Aspergillus niger stock inoculum. FIG. 29 PANELS B-F show the quantification of Aspergillus niger treated with different compositions (PANEL B: Control, PANEL C: PG, PANEL D: PG+MG1, PANEL E: PG+1:10 MG1, PANEL F: PG+1:100 MG1). The microbial quantification results are shown in Table 9. PG did not kill Aspergillus niger. Treatment with PG+MG1 resulted in full kill of Aspergillus niger. Treatment with PG+1:10 MG1 resulted in nearly full kill of Aspergillus niger. Treatment with PG+1:100 MG1 had similar effect compared with treatment with PG.

TABLE 9
Microbial quantification results

| | Testing sample | | | | | |
|---|---|---|---|---|---|---|
| | microbial concentration | | | | | |
| | (1:100 dilution) | | | | PG + 1:10 | PG + 1:100 |
| Organism | CFU/mL | Control | PG | PG + MG1 | MG1 | MG1 |
| S. aureus | 2.6 × 10⁶ | 2.2 × 10⁶ | 2.5 × 10⁴ | 0 | 0 | 0 |
| ATCC | | | | | | |
| 29213 | | | | | | |
| P. aeurginosa | 1.08 × 10⁹ | 4.6 × 10⁶ | 5.4 × 10⁵ | 0 | | |
| ATCC | | | | | | |
| 27853 1 | | | | | | |
| P. aeurginosa | 2.4 × 10⁶ | 2.3 × 10⁵ | 1.4 × 10⁵ | | 0 | 3.0 × 10⁵ |
| ATCC | | | | | | |
| 27853 2 | | | | | | |
| E. coli | 3.5 × 10⁶ | 2.7 × 10⁸ | 0 | 0 | 0 | 0 |
| C. albicans | 3.2 × 10⁵ | 2.0 × 10⁵ | Bacterial | 0 | | |
| ATCC | | | contaminated | | | |
| 26555 1 | | | | | | |
| C. albicans | 2.6 × 10⁶ | 2.2 × 10⁶ | 2.5 × 10⁴ | | 0 | 6 × 10² |
| ATCC | | | | | | |
| 26555 2 | | | | | | |
| B. cepacia | 5.3 × 10⁶ | 2.8 × 10⁸ | 4.9 × 10⁶ | 0 | 0 | 2.1 × 10⁵ |
| ATCC | | | | | | |
| 10856 | | | | | | |
| K. pneumonia | 4.8 × 10⁶ | 2.3 × 10⁶ | 0 | 0 | 0 | 0 |
| ATCC | | | | | | |
| BAA-2342 | | | | | | |
| E. cloacae | 5.1 × 10⁶ | 2.3 × 10⁶ | 0 | 0 | 0 | 0 |
| ATCC | | | | | | |
| BAA-2341 | | | | | | |
| Aspergillus | 7 × 10⁵ | 6 × 10⁵ | 6 × 10⁵ | 0 | 6 × 10³ | 8 × 10⁵ |
| niger | | | | | | |
| Pooled | 2.5 × 10⁸ | 4.0 × 10⁶ | 4.3 × 10⁵ | 0 | 0 | 4.9 × 10⁵ |
| inoculum 1 | | | | | | |
| Pooled | 2.5 × 10⁸ | 3.6 × 10⁶ | 4.1 × 10⁵ | 0 | 0 | 6.5 × 10⁵ |
| inoculum 2 | | | | | | |

Table 10 shows a summary of testing results.

TABLE 10
Summary of Testing results

| | Starting Cell | | | Log-reduction | Log-reduction |
|---|---|---|---|---|---|
| | Concentration for | Log-reduction | Log-reduction | with | with |
| | Treatment | with | with | PG + 1:10 | PG + 1:100 |
| Organism | (CFU/mL) | PG alone | PG + MG1 | MG1 | MG1 |
| S. aureus ATCC | 2.6 × 10⁶ | 2-logs | Full kill (6- | Full kill (6- | Full kill (6- |
| 29213 | | | logs) | logs) | logs) |
| P. aeurginosa | 2.4 × 10⁶ | 1-log | Full kill (6- | Full kill (6- | 1-log |
| ATCC 27853 | | | logs) | logs) | |
| E. coli | 3.5 × 10⁶ | Full kill (6- | Full kill (6- | Full kill (6- | Full kill (6- |
| | | logs) | logs) | logs) | logs) |
| C. albicans ATCC | 2.6 × 10⁶ | 2-logs | Full kill (6- | Full kill (6- | 4-logs |
| 26555 | | | logs) | logs) | |
| B. cepacia ATCC | 5.3 × 10⁶ | 0-logs | Full kill (6- | Full kill (6- | 1-log |
| 10856 | | | logs) | logs) | |
| K. pneumonia | 4.8 × 10⁶ | Full kill (6- | Full kill (6- | Full kill (6- | Full kill (6- |
| ATCC BAA-2342 | | logs) | logs) | logs) | logs) |
| E. cloacae ATCC | 5.1 × 10⁶ | Full kill (6- | Full kill (6- | Full kill (6- | Full kill (6- |
| BAA-2341 | | logs) | logs) | logs) | logs) |
| Pooled inoculum | 2.5 × 10⁸ | 1-log | Full kill (8- | Full kill (8- | 1-log |
| | | | logs) | logs) | |
| Aspergillus niger | 7 × 10⁵ | 0-logs | Full kill (5- | 2-logs | 0-logs |
| | | | logs) | | |

Addition of MG1 to PG improves antimicrobial activity against S. aureus, P. aeruginosa, B. cepacia, C. albicans, A. niger, and a pooled inoculum. Addition of MG1 and 1:10 MG1 to PG fully killed S. aureus, P. aeruginosa, B. cepacia, C. albicans, A. niger, and the microorganisms in the pooled inoculum. The MicroorGONE formulations improve disinfectant activity of a general cleaning product against bacteria and yeast, including multi-drug resistant bacteria and yeast, and endogenous bacterial contaminant.

1. A composition comprising, in a mixture:
a) a cationic surfactant, wherein the cationic surfactant is a cetyltrimethylammonium halide;
b) a chelating agent, wherein the chelating agent is N,N-Dicarboxymethyl glutamic acid (GLDA) or a salt thereof;
c) a pH modifier; and
d) a solvent.

2. The composition of claim 1, wherein the cetyltrimethylammonium halide is cetyltrimethylammonium chloride.

3. The composition of claim 1, wherein the cetyltrimethylammonium halide is cetyltrimethylammonium bromide.

4. The composition of claim 1, wherein the cetyltrimethylammonium halide is cetyltrimethylammonium iodide.

5. The composition of claim 1, wherein the chelating agent is N,N-Dicarboxymethyl glutamic acid tetrasodium salt.

6. The composition of claim 1, wherein the cationic surfactant is present in the mixture at a concentration of about 0.2 mM to about 50 mM.

7. (canceled)

8. The composition of claim 1, wherein the cationic surfactant is present in the mixture at a concentration of about 2 mM.

9. The composition of claim 1, wherein the cationic surfactant is present in the mixture at a concentration of about 3 mM.

10. The composition of claim 1, wherein the chelating agent is present in the mixture at a concentration of about 1 mM to about 200 mM.

11. The composition of claim 1, wherein the chelating agent is present in the mixture at a concentration of about 20 mM.

12. The composition of claim 1, wherein the chelating agent is present in the mixture at a concentration of about 26 mM.

13. (canceled)

14. The composition of claim 1, wherein the pH modifier comprises acetic acid.

15. The composition of claim 1, wherein the pH modifier is present in the mixture at a concentration of about 1 mM to about 150 mM.

16. The composition of claim 1, wherein the pH modifier is present in the mixture at a concentration of about 10 mM.

17. The composition of claim 1, wherein the pH modifier is present in the mixture at a concentration of about 15.6 mM.

18. The composition of claim 1, wherein the solvent is present in the mixture in an amount of about 96 wt % to about 98.9 wt %.

19. The composition of claim 1, wherein the composition is substantially free of enzymes.

20. The composition of claim 1, wherein the composition is substantially free of oxidant.

21. The composition of claim 1, wherein the composition is substantially free of water-soluble film forming polymers.

22. The composition of claim 1, wherein the solvent is water.

23-89. (canceled)
